(19) United States
(12) Patent Application Publication
(10) Pub. No.: US 2024/0252599 A1
(43) Pub. Date: Aug. 1, 2024

(54) ALKALINE PHOSPHATASE-BASED TREATMENTS OF CELIAC DISEASE

(71) Applicant: Theriva Biologics, Inc., Rockville, MD (US)

(72) Inventor: Michael KALEKO, Rockville, MD (US)

(21) Appl. No.: 18/563,277

(22) PCT Filed: May 24, 2022
(86) PCT No.: PCT/US22/30653

§ 371 Date: Nov. 21, 2023

Related U.S. Application Data
(60) Provisional application No. 63/192,265, filed on May 24, 2021.

(51) Int. Cl. A61K 38/46

(52) U.S. Cl. CPC A61K 38/465; C12Y 301/03001

(57) ABSTRACT

The present disclosure relates, inter alia, to therapeutic intestinal alkaline phosphatases for the treatment of celiac disease.

### CROSS-REFERENCE TO RELATED APPLICATIONS

This application claims the benefit of U.S. Provisional Application No. 63/192,265, filed May 24, 2021, the entire contents of which are hereby incorporated by reference in their entirety.

### TECHNICAL FIELD

The present disclosure relates, inter alia, to therapeutic intestinal alkaline phosphatases for the treatment of celiac disease.

### DESCRIPTION OF THE TEXT FILE SUBMITTED ELECTRONICALLY

The content of the text file submitted electronically herewith is incorporated herein by reference in their entirety: A computer readable format copy of the Sequence Listing (Filename: “SYN-054PC_SequenceListing.txt”; Date created: May 23, 2022; File size: 45 KB).

### BACKGROUND

Celiac disease is one of the most common autoimmune disorders and is prevalent in both the United States and European Union at an estimated rate of 0.5% to 1.0% of the general population, including children and adults. Celiac disease; an autoimmune disease, is triggered in genetically-predisposed individuals by ingestion of gluten proteins from wheat as well as other grains. Active celiac disease is associated with damaged villi and low intestinal alkaline phosphatase (IAP) levels, and disease pathology is associated with barrier dysfunction and inflammation. While celiac disease presentation and course can vary from patient to patient, the underlying causes are generally the same. Specifically, gluten-derived peptides open the gut barrier, leak into the intestinal wall, stimulate an inflammatory response, and then cause an autoimmune reaction to human proteins, most notably tissue transglutaminase.

Alkaline phosphatase (“APs,” EC 3.1.3.1) is a hydrolase enzyme that can remove phosphate groups from various targets, including nucleotides and proteins. In particular, mammalian APs exert their properties by primarily targeting LPS (a TLR4 agonist), flagellin (a TLR5 agonist) and CpG DNA (a TLR9 agonist). APs also degrade intestine luminal NTPs (e.g., ATP, GTP, etc.), which promote the growth of good bacteria and reverses dysbiosis. Accordingly, APs may find clinical use as, for example, microbiome preserving agents for treating various gastrointestinal (GI) disorders.

Currently, with no pharmaceutical treatments available, the only effective treatment for celiac disease is a restrictive gluten-free diet for life. Accordingly, there is a large unmet medical need for therapies directed to the treatment and prevention of celiac disease.

### SUMMARY

Accordingly, in some aspects, the present disclosure provides intestinal alkaline phosphatase (IAP) constructs, including variants thereof, for the treatment and/or prevention of celiac disease. In embodiments, the IAP construct is a mammalian IAP including, but not limited to, human IAP (hIAP), calf IAP (cIAP), and bovine IAP (bIAP). In embodiments, the IAP is secreted from the host cell. In embodiments, the IAP and is administered orally.

In another aspect, the present disclosure provides methods for treating or preventing celiac disease via administration of a therapeutic IAP described herein.

The present disclosure provides methods for treating celiac disease, including in conjunction with a gluten-free diet (GFD). The disclosure provides methods for treating symptomatic or active celiac disease, despite reasonable or substantial compliance with a GFD. In embodiments, the disclosure provides methods of treating patients having refractory celiac disease or celiac disease that is non-responsive to a GFD. The methods comprise administering a pharmaceutical composition comprising an IAP, or modified version thereof. Administration of IAP promotes GI tight junction integrity. When administered to the GI, for example, IAP can reduce or ameliorate symptomatic, active, refractory, and/or non-responsive celiac disease.

As disclosed herein, IAP is safe and effective for prolonged use, and in embodiments, celiac disease symptoms (including GI symptoms, abdominal symptoms, and non-GI symptoms) continue to decline with prolonged use of IAP. For example, in embodiments, the composition comprising IAP is administered to the celiac disease patient about 2 or 3 times per day for at least about 8 weeks. In embodiments, the composition is administered for at least about 9 weeks, at least about 10 weeks, or at least about 12 weeks, or more, such as for at least about 6 months or for at least about 1 year. The composition may be administered prior to meals, to promote tight junction integrity in the GI in case of unintentional exposure to gluten or intentional exposure of small amounts of gluten.

In embodiments, at the start of treatment with IAP the celiac disease patient may be experiencing one or more classes of symptoms, such as abdominal domain symptoms, diarrhea and loose stools domain symptoms, nausea domain symptoms, gastrointestinal domain symptoms, and non-GI domain symptoms, despite being on a GFD. In embodiments, the patient experiences non-GI domain symptoms together with abdominal domain symptoms, diarrhea and loose stools symptoms, nausea symptoms, and/or GI domain symptoms, despite reasonable or substantial compliance with a GFD. Exemplary celiac symptoms include a plurality of abdominal cramping, abdominal pain, bloating, gas, diarrhea, loose stools, nausea, vomiting, indigestion, reflux, constipation, vomiting, headache, and tiredness. Prior to treatment, the patient may experience at least about 2, or at least about 3, at least about 4, at least about 5, at least about 6, or at least about 7 CeD PRO symptomatic days per week, which is reduced by one or more days upon treatment with IAP for at least about 8 weeks. For example, upon treatment with IAP the patient experiences one, two or more CeD PRO improved symptom days (or non-symptomatic days) per week, and over time symptoms may substantially or entirely subside.

### BRIEF DESCRIPTION OF THE DRAWINGS

FIG. 1A-D depicts sequences pertaining to alkaline phosphatase agents present in compositions and/or formulations described herein.

### DETAILED DESCRIPTION

The role of alkaline phosphatases (APs) in promoting growth of good bacteria and reversing dysbiosis is a significant and growing field of study in the advancement of treatment options for disorders like celiac disease, where the consensus is that broad exposure to commensal, non-pathogenic microorganisms early in life is associated with protection against celiac disease.

In particular, intestinal alkaline phosphatase (IAP) is an endogenous protein expressed by the intestinal epithelium that can be used to mitigate inflammation and maintain gut homeostasis. For example, loss of IAP expression or function is associated with increased intestinal inflammation, dysbiosis, bacterial translocation, and systemic inflammation. Its primary functions, among others, in maintaining intestinal homeostasis are generally recognized as the regulation of bicarbonate secretion and duodenal surface pH, long chain fatty acid absorption, mitigation of intestinal inflammation through detoxification of pathogen-associated molecular patterns, and regulation of the gut microbiome. Further, IAP acts directly on the cells that line the GI tract to improve barrier function and diminish so called “leaky gut.” Several substrates that are acted on by IAP's phosphatase functions include lipopolysaccharide (LPS), flagellin, CpG DNA, and nucleotide di- and tri-phosphates. Specifically, IAP is a target for therapeutics due to its ability to downregulate inflammation, regulate the microbiome, tighten the gut barrier through enhanced expression of claudins and occludins, and affect metabolism of adenosine tri-phosphate and diphosphate (ATP and ADP).

The present disclosure is directed, in part, to pharmaceutical compositions, formulations, and uses of intestinal alkaline phosphatases (IAPs) for the treatment and/or prevention of celiac disease.

### Celiac Disease and Methods of Treatment

Celiac disease (CD) is a chronic autoimmune disease that is HLA-DQ2/DQ8 haplotype restricted. Glutens, the major protein fraction of wheat, and related proteins in rye and barley are the triggering agents of the disease. Ingested gluten or its derivative fractions (gliadin and subunits) elicit a harmful T cell-mediated immune response after crossing the small bowel epithelial barrier, undergoing deamidation by tissue transglutaminase (tTG) and engaging class II MHC molecules.

While the earliest events leading to CD involve innate immune responses, evidence in the literature seems to suggest that a dysfunctional cross talk between innate and adaptive immunity is also an important pathogenic element in the autoimmune process of the disease. Under physiological circumstances, the intestinal epithelium, with its intact intercellular tight junctions, serves as a key barrier to the passage of macromolecules such as gluten. When the integrity of the tight junction system is compromised, as in CD, a paracellular leak (“leaky gut”) and an inappropriate immune response to environmental antigens (i.e., gluten) may develop.

In celiac intestinal tissues and in in vitro, ex vivo, and in vivo animal experiments, gluten/gliadin causes a rapid increase in permeability in normal and diseased states. Animal models likewise have demonstrated the association of gluten, increased paracellular permeability and other autoimmune diseases, including type 1 diabetes (T1D).

Celiac disease patients can be identified, diagnosed or confirmed, for example, by measuring the serum levels of anti-endomysial antibody, anti-tissue transglutaminase antibody (anti-tTG), and/or anti-deamidated gliadin peptide (anti-DGP). Celiac patients may also be diagnosed, for example, by small bowel biopsy and/or capsule endoscopy. In addition, patients can be screened for genes encoding the human leukocyte antigens HLA-DQ2 and HLA-DQ8, which are statistically associated with celiac disease. Furthermore, in embodiments, patients exhibiting villous atrophy are identified as having celiac disease via screening for anti-endomysial (EMA) antibodies of the immunoglobulin A (IgA) type.

Without wishing to be bound by theory, patients with active or refractory CD have low endogenous IAP levels, e.g. relative to an untreated or undiseased patient, and thus would benefit from IAP supplementation. In embodiments, the patient has active CD associated with low endogenous IAP levels, e.g. relative to an untreated or undiseased patient. In embodiments, the patient has refractory CD associated with low endogenous IAP levels. In embodiments, the patient has low expression and/or activity of IAP in the patient's mucosa. In embodiments, the patient has low expression and/or activity of IAP in the patient's mucosa. In embodiments, the patient has low expression and/or activity of IAP in the patient's intestinal mucosa. In embodiments, the patient has low expression and/or activity of IAP in the patient's colonic mucosa. In embodiments, the patient is characterized as having low expression and/or activity of IAP by assaying a biological sample from the subject. In embodiments, the biological sample is selected from stool, mucus, tissue, blood, plasma, serum, pus, urine, perspiration, tears, sputum, saliva, and/or other body fluids. In embodiments, the biological sample is stool. 20 In embodiments, low expression of IAP is less than about 30 U/L, or less than about 25 U/L, less than about 20 U/L, less than about 10 U/L, less than about 5 U/L, less than about 1 U/L. In embodiments, low expression of IAP is less than about 30 U/L, or less than about 25 U/L, less than about 20 U/L, less than about 10 U/L, less than about 5 U/L, less than about 1 U/L in patient blood.

In aspects, the disclosure provides a diagnostic method for monitoring histological recovery of the patient comprising assaying a biological sample from the patient for expression and/or activity of IAP in the patient's mucosa. In an aspect, the disclosure provides a diagnostic method for monitoring histological recovery of the patient comprising assaying a biological sample from the patient for expression and/or activity of IAP in the patient's intestinal mucosa. In aspects, the disclosure provides a diagnostic method for monitoring histological recovery of the patient comprising assaying a biological sample from the patient for expression and/or activity of IAP in the patient's colonic mucosa. In aspects, the disclosure provides a companion diagnostic method for selecting a patient suitable for treatment with the present IAP-based agent, comprising assaying a biological sample from the patient for expression and/or activity of IAP in the patient's mucosa, before or after administration of the present IAP-based agent. In embodiments, the biological sample is selected from stool, mucus, tissue, blood, plasma, serum, pus, urine, perspiration, tears, sputum, saliva, and/or other body fluids. In embodiments, the biological sample is stool. In embodiments, the expression and/or activity of IAP is assessed relative to an untreated or undiseased patient.

In embodiments, patients are selected for treatment with a composition comprising IAP, as described herein, via the Marsh-Oberhuber histological classification, which is divided into various types: Type 1 patients exhibit infiltrative lesions; Type 2 patients exhibit crypt hyperplasia; Type 3A: Partial patients exhibit shortened blunt villi, infiltration intraepithelial lymphocytes (IEL), and hyperplastic crypts; Type 3B: Subtotal patients exhibit recognizable atrophic villi, inflammatory cells, and enlarged crypts; and Type 3C: Total patients exhibit total villous atrophy (total absence of villi) and severe atrophic, hyperplastic, and infiltrative lesions.

In embodiments, patients are selected for treatment with a composition comprising IAP, as described herein, via the Corazza Villanacci histological classification, which is divided into various Grades: Grade A patients exhibit no atrophy, normal villous architecture with or without crypt hyperplasia and greater than or equal to 25 IELs/100 enterocytes; Grade B1 patients exhibit a villous-crypt ratio less than 3 to 1 and an IEL count of greater than 25/100 enterocytes; and Grade B2 patients exhibit completely flat mucosa with IE lymphocytosis and crypt hyperplasia.

In embodiments, patients are selected for treatment with a composition comprising IAP, as described herein, when they test positive for the “four out of five rule,” which indicates that four out of five of the following criteria must be met to establish a CD diagnosis: (1) typical signs and symptoms (e.g., diarrhea and/or malabsorption); (2) antibody positivity; (3) HLA-DQ2 and/or HLA/DQ8 positivity; (4) intestinal damage (e.g., villous atrophy and/or minor lesions); and (5) clinical response to GFD.

In embodiments, celiac disease is diagnosed more frequently in females than in males, according to serological screening. In embodiments, celiac disease occurs at any age, optionally wherein the patient is in early childhood or elderly.

In embodiments, the patient has classic, non-classic, subclinical, potential or refractory celiac disease, according to the Oslo classification. In embodiments, the celiac disease patient has the intestinal (e.g., classic) form of CD. The intestinal form of CD is more commonly detected in the pediatric population and children younger than 3 years and is characterized by diarrhea, loss of appetite, abdominal distention, and failure to thrive. In embodiments, older children and adults exhibit diarrhea, bloating, constipation, abdominal pain, or weight loss. In embodiments, the intestinal form of CD is associated with hospitalization due to cachexia, sarcopenia, significant hypoalbuminemia, and electrolyte abnormalities. In embodiments, the intestinal form of CD is associated with an irritable bowel syndrome-like presentation with constipation or alternating bowel and/or dyspepsia-like symptoms, including nausea and vomiting. In embodiments, treatment of a patient (e.g., a child younger than 3 years) having the intestinal form of CD with a composition comprising an IAP, as described herein, results in a reduction of one or more of diarrhea, loss of appetite, abdominal distention, and failure to thrive. In further embodiments, treatment of a patient (e.g., an older child or adult) having the intestinal form of CD with a composition comprising an IAP, as described herein, results in a reduction of one or more of diarrhea, bloating, constipation, abdominal pain, or weight loss.

In embodiments, the celiac disease patient has the extraintestinal (e.g., non-classic) form of CD. Extraintestinal symptoms are common in both children and adults. In embodiments, symptoms of the extraintestinal form of CD include, but are not limited to, iron deficiency microcytic anemia, detectable in up to 40% of cases (by cause of iron malabsorption or chronic inflammation) or, more rarely, macrocytic anemia due to folic acid and/or vitamin B12 deficiency (more frequent in Europe than in the US). In embodiments, patients having the extraintestinal form of CD exhibit changes in bone mineral density, including osteopenia or osteoporosis (affecting about 70% of patients at diagnosis) due to altered absorption of calcium and vitamin D3. In embodiments, some patients (e.g., children) having the extraintestinal form of CD exhibit growth retardation, short stature, tooth enamel defects, aphthous stomatitis, and/or hypertransaminasemia. In further embodiments, patients having the extraintestinal form of CD exhibit neurological symptoms, including, but not limited to, headache, paresthesia, neuroinflammation, anxiety, and/or depression. In embodiments, patients having the extraintestinal form of CD exhibit changes in reproductive function, including, but not limited to, late menarche, amenorrhea, recurrent miscarriages, premature birth, early menopause, and/or changes in the number and mobility of spermatozoa.

The subclinical form of CD includes patients with symptoms/signs below the clinical identification threshold and are often recognizable only after the appreciation of the beneficial effects induced by the GFD. In embodiments, a patient having the subclinical form of CD undergoes antibody screening due to being a relative of a CD patient or identified as a result of a screening strategy in the general population. In embodiments, patients having the subclinical form of CD exhibit no symptoms.

The potential form of CD is characterized by positive serological and/or genetic markers with normal intestinal mucosa and minimal signs of inflammation such as increase in intraepithelial lymphocytes (IELs). In embodiments, a patient having the potential form of CD manifest classic and/or non-classic symptoms or are entirely asymptomatic.

In embodiments, celiac disease is associated with other concomitant diseases/disorders, including, but not limited to, autoimmune and/or idiopathic diseases, including, but not limited to, dermatitis herpetiformis, type 1 diabetes mellitus, Hashimoto's thyroiditis, Graves' disease, selective IgA deficiency, vitiligo, alopecia areata, psoriasis, Addison's disease, autoimmune atrophic gastritis, and/or IgA nephropathy (i.e., Berger's disease); connective tissue diseases, including, but not limited to, Sjogren's syndrome, scleroderma, systemic erythematosus lupus, polymyositis, rheumatoid arthritis, and/or myasthenia gravis; chromosomal diseases, including, but not limited to, Down syndrome, Turner syndrome, or William's syndrome; neurological diseases, including, but not limited to, cerebellar ataxia, peripheral neuropathy, multiple myoclonic seizures, multiple sclerosis, cerebral atrophy, or epilepsy with and without occipital calcifications; hepatic autoimmune diseases, including, but not limited to, primary biliary cholangitis; and/or idiopathic diseases, including, but not limited to, dilated cardiomyopathy, sarcoidosis, and atopy. In embodiments, celiac disease is associated with one or more conditions selected from microscopic colitis, irritable bowel syndrome, small intestine bacterial overgrowth, lactose/fructose intolerance, diverticular disease, Crohn's disease, pancreatic insufficiency, and drug-induced enteropathy. In embodiments, treatment of a patient having celiac disease and one or more concomitant diseases with a composition comprising IAP results in resolution of symptoms, prevention of complications, and/or improvement of the associated disease.

In embodiments, administration of IAP treats, or prevents the development of, autoimmune disease associated with celiac disease. In embodiments, administration of IAP treats one or more autoimmune diseases associated with celiac disease selected from type 1 diabetes mellitus, Hashimoto's thyroiditis, Graves' disease, autoimmune hepatitis, primary biliary cholangitis, dermatitis herpetiformis, vitiligo, Addison's disease, alopecia, psoriasis, IgA deficiency, autoimmune atrophic gastritis, autoimmune hemolytic anemia, Sjogren's syndrome, scleroderma, systemic erythematosus lupus, polymyositis, rheumatoid arthritis, myasthenia gravis, and/or IgA nephropathy (i.e., Berger's disease).

In embodiments, administration of IAP treats, or prevents the development of, cancer (e.g., an intestinal lymphoma) associated with celiac disease. In embodiments, administration of IAP treats one or more cancers associated with celiac disease selected from Hodgkin T cell intestinal lymphoma, B cell lymphoma, and small bowel adenocarcinoma.

In embodiments, administration of IAP treats, or prevents the development of, complications associated with celiac disease. For example, in embodiments, administration of IAP treats one or more diseases selected from hyposplenism, refractory celiac disease (RCD), intestinal lymphoma, small bowel adenocarcinoma, and ulcerative jejunoileitis.

In embodiments, administration of IAP reduces the likelihood of a shortened lifespan associated with celiac disease.

In embodiments, administration of IAP treats, or prevents the development of, brain lesions (e.g., white-matter lesions) associated with celiac disease. In embodiments, administration of IAP treats, or prevents the development of, neurological complications of brain lesions (e.g., white-matter lesions) associated with celiac disease selected from epilepsy, occipital calcifications, folate deficiency, and cerebellar ataxia.

In embodiments, administration of IAP treats, or prevents the development of, diseases and/or disorders associated with celiac disease. For example, in embodiments, administration of IAP treats diseases and/or disorders associated with celiac disease selected from diarrhea, weight loss, malabsorption, constipation, anaemia, osteoporosis, hypertransaminasemia, neurological disorders, and dermatitis herpetiformis.

In embodiments, administration of IAP treats, or prevents the development of, gluten-induced or gluten-correlated cognitive impairment (“brain fog”) associated with celiac disease. In embodiments, administration of IAP treats, or prevents the development of, one or more transient cognitive impairments to memory, attention, executive function, and the speed of cognitive processing associated with celiac disease.

In embodiments, the celiac disease patient may not be reasonably or substantially compliant with a gluten-free diet (GFD), meaning that gluten exposure is not merely of an unintentional nature. In accordance with the disclosure, the celiac symptoms of these patients can be reduced, ameliorated, or prevented, thereby allowing for some GFD non-compliance. In embodiments, the celiac disease patient is reasonably or substantially compliant with a gluten-free diet, meaning that any significant gluten exposure is inadvertent or infrequent. For example, in embodiments, prior to treatment with IAP the celiac disease patient has been on a GFD for at least about 1 week, at least about 2 weeks, at least about 3 weeks, at least about 4 weeks, at least about 1 month, at least about 2 months, at least about 3 months, at least about 4 months, at least about 5 months, or at least about 6 months. In embodiments, the celiac disease patient has been on a GFD for at least about 1 year, at least about 2 years, at least about 3 years, at least about 4 years, or at least about 5 years. Despite being on a GFD for a length of time, and despite reasonable compliance with the GFD, the patient may still experience celiac symptoms as described herein. IAP in conjunction with the GFD can reduce these symptoms, even when these symptoms are significant or substantial, and/or when even non-GI symptoms are experienced or prevalent.

In embodiments, the celiac disease is non-responsive to GFD. Patients with non-responsive celiac disease do not exhibit a histological response to a gluten-free diet. Such patients continue to exhibit small-bowel mucosal villous atrophy during a gluten-free diet which is diagnosed by intestinal biopsy. See Pulido et al., (2013) Can J. Gastroenterol, 27(8):449-453 and Spatoloa et al., (2014) Aliment Pharmacol Ther. 39(4): 407-417, the entire contents of which are hereby incorporated by reference. The most common reason for this lack of recovery despite being on gluten-free diet is continuing gluten ingestion (intentional and unintentional), because gluten is present in many foods and medications. For example, the patient may exhibit a high sensitivity to gluten. Other potential reasons include, for example, the development of other complications such as irritable bowel syndrome, small bowel bacterial overgrowth, other food intolerances such as lactose intolerance, microscopic colitis, Crohn's disease, ulcerative colitis, and pancreative digestive enzyme insufficiency.

Accordingly, in embodiments, the present disclosure treats celiac disease patients who continue to be exposed to gluten, intentionally and/or unintentionally. In embodiments, the celiac disease patient is non-responsive to a GFD. In embodiments, the patient is determined to have irritable bowel syndrome, small bowel bacterial overgrowth, or other food intolerances such as lactose intolerance, gastroesophageal reflux, microscopic colitis, Crohn's disease, ulcerative colitis, and pancreative digestive enzyme insufficiency. In further embodiments, the present disclosure reduces symptoms in celiac disease patients having, for example, extra-intestinal diseases and/or conditions including, but not limited to, dermatitis herpetiformis, diabetes (Type 1 and 2), autoimmune thyroid disease, anemia, dental enamel hypoplasia/tooth discoloration, osteopenia or osteoporosis, abnormal liver function tests, joint pain and/or join disease, and recurrent miscarriages or fertility problems. These non-GI conditions may be ameliorated, avoided, or managed in part with an IAP regimen as described herein.

In embodiments, the present disclosure provides methods for treating celiac disease patients having refractory celiac disease. Refractory celiac disease is defined by persistent or recurrent malabsorptive symptoms and damaged intestinal architecture despite strict adherence to a gluten-free diet for at least six to twelve months in the absence of other causes of non-responsive celiac disease and overt malignancy. See Spatoloa et al., (2014) Aliment Pharmacol Ther. 39(4): 407-417, Rubio-Tapia et al., Gut, 59(4):547-557, and Semrad (2008) Impact, 8(3): 1-3, the entire contents of which are hereby incorporated by reference. In embodiments, refractory CD is characterized by persistent symptoms and atrophy of the intestinal villi after at least twelve months of a strict GFD. Some of these patients never respond to a gluten-free diet while others initially respond but have a recurrence of symptoms and intestinal inflammation. Most patients with refractory celiac disease have persistent diarrhea, abdominal pain, malabsorption, and involuntary weight loss in addition to vitamin and mineral deficiencies, anemia, fatigue, and malaise. Refractory celiac disease is divided into two types. Type I patients exhibit normal T cell population in the intestinal lining and are conventionally treated with aggressive nutritional support as well as pharmacologic therapies including steroids. In contrast, type II patients show abnormal T-cell population in the intestinal lining. These patients have a poor prognosis as they respond poorly to steroid treatment and have a high chance of developing severe complications such as enteropathy-associated T-cell lymphoma (EATL), collagenous sprue, and ulcerative jejunitis. Accordingly, in embodiments, the present disclosure provides methods for treating celiac disease patient with type I refractory celiac disease. In embodiments, the present disclosure provides methods for treating celiac disease patient with type II refractory celiac disease. In embodiments where the patient has non-responsive or refractory celiac disease, the patient may undergo an adjunct therapy. Exemplary adjunct therapy includes treatment with any of the additional therapeutic agents as described herein. For example, the celiac disease patient may undergo an adjunct therapy involving an anti-inflammatory such as steroid treatment (e.g., prednisone, budesonide, prednisolone, etc.) or NSAID treatment. Alternatively, the celiac disease patient may undergo adjunct therapy with immunosuppressants and other biological modifiers such as azathioprine, cyclosporin, infliximab and alemtuzumab treatment. Alternatively, the celiac patient may undergo therapy with an antibiotic to control bacterial overgrowth in the GI. Alternatively or in addition, the patient may undergo treatment with a probiotic.

In embodiments, a patient has seronegative celiac disease. In embodiments, the seronegative form is characterized by the lack of demonstrable serological markers along with clinical signs of severe malabsorption and atrophy of the intestinal mucosa. In embodiments, a patient has GFD non-responsive celiac disease. Non-responsive celiac disease indicates gastrointestinal symptoms that persist despite following a gluten-free diet of more than twelve months.

In embodiments, the celiac disease patient is experiencing symptoms, despite substantial compliance with GFD, or due to non-compliance. While symptoms may be determined by the attending physician through examination/interview of the patient, there are various tools for quantifying or evaluating a patient's symptoms, well-being, and GFD compliance, which may also be employed. These include, but are not limited to, Celiac Disease Patient Reported Outcome (CeD PRO), Gastrointestinal Symptom Rating Scale (GSRS), Celiac Disease Gastrointestinal Symptom Rating Scale (CeD GSRS), Bristol Stool Form Scale (BSFS), General Well-Being Questionnaire, Short Form 12 Health Survey Version 2 (SF12V2), Celiac Disease Quality of Life Questionnaire (CeD-QoL), and Clinician Global Assessment of Disease Activity (CGA).

Adherence to gluten-free diet may be assessed by, for example, Celiac Dietary Adherence Test (CDAT) and Gluten-Free Diet Compliance Questionnaire (GFDCQ). Accordingly, in embodiments, the celiac disease patients are experiencing one or more symptoms as measured by one of the scales described herein.

In embodiments, the celiac disease patient is experiencing one or more symptoms as measured by CeD PRO at the start of treatment with IAP. The CeD PRO questionnaire was developed to assess symptom severity in clinical trials in subjects with celiac disease. Items in the questionnaire were formulated based on one-on-one interviews with subjects with celiac disease and thus reflect the symptoms that subjects consider part of their celiac disease experience. The CeD PRO includes 12 items asking participants about the severity of celiac disease symptoms they experience each day. Subjects rate their symptom severity on an 11-point, 0 to 10 scale; from “not experiencing the symptom” to “the worst possible symptom experience”. The CeD PRO included a Gastrointestinal Domain scale consisting of all the gastrointestinal symptoms (abdominal cramping, abdominal pain, bloating, constipation, diarrhea, gas, loose stools, nausea, vomiting), and a Non-gastrointestinal Domain scale which include items such as headache and tiredness. Each domain score is calculated by summing the value of the individual items within a scale and averaging across the number of items within the scale. Higher scores reflect greater symptom severity.

In embodiments, the celiac disease patient is experiencing one or more CeD PRO abdominal domain symptoms selected from abdominal pain, abdominal cramping, bloating or gas. In embodiments, the celiac disease patient is experiencing one or more CeD PRO Gastrointestinal (GI) domain symptoms selected from abdominal cramping, abdominal pain, bloating, gas, diarrhea, loose stools, nausea, indigestion, reflux, constipation, and vomiting. In embodiments, the celiac disease patient is experiencing one or more of diarrhea and loose stools. In embodiments, the celiac disease patient is experiencing nausea. In embodiments, the celiac disease patient is experiencing constipation. In embodiments, the celiac disease patient is experiencing vomiting. In embodiments, the celiac disease patient is experiencing one or more CeD PRO Non-Gastrointestinal domain symptoms including, but not limited to, headaches and tiredness. The patient may be experiencing one or more CeD PRO Non-GI domain symptoms, with one or more CeD PRO abdominal domain or CeD PRO GI domain symptoms.

In embodiments, the celiac disease patient is experiencing one or more significant or severe symptomatic days (based on CeD PRO) at the start of treatment with IAP. A CeD PRO Symptomatic day can include abdominal domain and GI domain symptoms, and/or non-GI domain symptoms. A CeD PRO Symptomatic day is defined as a day where the mean of the set of symptoms is scored as greater than or equal to 2.5 out of a 0 to 10 scale, or greater than or equal to 3 out of a 0 to 10 scale. In embodiments, the celiac disease patient is experiencing one or more CeD PRO Symptomatic days of at least 2, 3, 4, 5, 6, or 7. CeD PRO abdominal domain symptoms are abdominal pain, abdominal cramping, bloating or gas. CeD PRO GI domain symptoms and/or one or more CeD PRO GI domain symptoms are abdominal cramping, abdominal pain, bloating, gas, diarrhea, loose stools, nausea, indigestion, reflux, constipation, and vomiting. CeD PRO non-GI domain symptoms include headache or tiredness. An additional non-GI symptom can include dermatitis herpetiformis.

In embodiments, the celiac disease patient is experiencing one or more symptoms as rated by the Gastrointestinal Symptom Rating Scale (GSRS) at the start of treatment with IAP. The GSRS is a 15-question, 7-scale questionnaire to assess 5 dimensions of gastrointestinal syndromes: diarrhea, indigestion, constipation, abdominal pain, and reflux. The questionnaire was originally constructed to measure symptoms in subjects with irritable bowel syndrome and peptic ulcer. In embodiments, the patient scores at least 2, or at least 3, or at least 4, or at least 5, or at least 6, or 7 on the Gastrointestinal Symptom Rating Scale (GSRS) at the start of treatment with IAP using the 1-7 Likert scale, with 1 representing the most positive option and 7 the most negative option. In embodiments, the celiac disease patient is experiencing one or more symptoms as rated by the Celiac Disease Gastrointestinal Symptom Rating Scale (CeD GSRS) at the start of treatment with IAP. The CeD GSRS dimensions measure a subset of the GSRS with dimensions more applicable to celiac disease. The CeD GSRS dimensions include 10 questions in the following domains: Diarrhea syndrome; Indigestion syndrome; and Abdominal Pain syndrome. In embodiments, the patient scores at least 2, or at least 3, or at least 4, or at least 5, or at least 6 or at least 7 on the CeD Gastrointestinal Symptom Rating Scale (CeD GSRS) at the start of treatment with IAP using the 1-7 Likert scale, with 1 representing the most positive option and 7 the most negative option.

In embodiments, the celiac disease patient is experiencing diarrhea and loose bowel movements at the start of treatment with IAP. In embodiments, the celiac disease patient is experiencing at least about 3, about 4, about 5, about 6, about 7, about 8, about 9, about 10, or more bowel movements per day at the start of treatment with IAP. In embodiments, the celiac disease patient is experiencing more than about 12, more than about 14, more than about 16, more than about 18 bowel movements per day at the start of treatment with IAP.

The Bristol Stool Form Scale (BSFS) is a pictorial aid to help patients identify the shape and consistency of their bowel movements. For example, the BSFS differentiates stools into seven types: Type 1: Separate hard lumps, like nuts (hard to pass); Type 2: Sausage-shaped, but lumpy; Type 3: Like a sausage but with cracks on its surface; Type 4: Like a sausage or snake, smooth and soft; Type 5: Soft blobs with clear cut edges (passed easily); Type 6: Fluffy pieces with ragged edges, a mushy stool; Type 7: Watery, no solid pieces, entirely liquid. Types 1-2 indicate constipation, with 3 and 4 being the ideal stools (especially the latter), as they are easy to defecate while not containing any excess liquid, and 5, 6 and 7 tending towards diarrhea. In embodiments, the celiac disease patient is experiencing at least about 4, at least about 5, at least about 6, at least about 7, or at least about 8, or at least about 9, or at least about 10 diarrhea or loose stools per day on the Bristol Form Scale (BSFS) at the start of treatment IAP. In embodiments, the celiac disease patient is experiencing 3 diarrhea or loose stools per day with a score of 5-7 as measured by the Bristol Form Scale (BSFS) at the start of treatment with IAP.

In embodiments, administration of IAP effectively improves the symptoms and sense of well-being of celiac disease patients. Improvements can be assessed using the various scales as described herein, or be determined by the attending physician by patient evaluation. For example, improvements in symptoms and sense of well-being may be evaluated by, but not limited to, CeD PRO, GSRS, CeD GSRS, BSFS, General Well-Being Question, SF12V2, CeD-QoL, and CGA scores.

In embodiments, administration of IAP results in a reduction of the CeD GSRS score, that is, administration of IAP results in a reduction in symptoms as measured by a change from baseline in CeD GSRS score. For example, administration of IAP may reduce the CeD GSRS score by at least about 20%, at least about 30%, at least about 40%, at least about 50%, at least about 60%, at least about 70%, at least about 75%, at least about 80%, at least about 85%, at least about 90%, at least about 95%, or about 100%.

In embodiments, administration of IAP results in a reduction of the CeD PRO abdominal domain score. In embodiments, administration of IAP results in a reduction in symptoms as measured by a change from baseline in CeD PRO abdominal domain score. For example, administration of IAP may reduce the CeD PRO abdominal domain score by at least about 20%, at least about 30%, at least about 40%, at least about 50%, at least about 60%, at least about 70%, at least about 75%, at least about 80%, at least about 85%, at least about 90%, at least about 95%, or about 100%.

In embodiments, administration of IAP results in a reduction of the CeD PRO gastrointestinal domain score. For example, administration of IAP may reduce the CeD PRO gastrointestinal domain score by at least about 20%, at least about 30%, at least about 40%, at least about 50%, at least about 60%, at least about 70%, at least about 75%, at least about 80%, at least about 85%, at least about 90%, at least about 95%, or about 100%.

In embodiments, administration of IAP results in a reduction of the CeD PRO Non-Gastrointestinal domain score (headache and tiredness). For example, administration of IAP may reduce the CeD PRO Non-Gastrointestinal domain score by at least about 20%, at least about 30%, at least about 40%, at least about 50%, at least about 60%, at least about 70%, at least about 75%, at least about 80%, at least about 85%, at least about 90%, at least about 95%, or about 100%.

In embodiments, administration of IAP results in an increase in CeD PRO Improved Symptom days. A CeD PRO Improved Symptom day is a day where the mean of abdominal cramping, abdominal pain, bloating, and gas is scored as less than or equal to 1.5 out of a 0 to 10 scale, and a day where the mean of diarrhea and loose stool is scored as less than or equal to 1.5 out of a 0 to 10 scale, and a day where nausea is scored as less than or equal to 1 out of a 0 to 10 scale. For example, administration of IAP increases the number of (e.g., average weekly number of) CeD PRO Improved Symptom days by at least about 1, 2, 3, 4, 5, 6, or 7 days.

In embodiments, administration of IAP results in the patient experiencing a reduction in CeD PRO Symptomatic days (e.g., average weekly number of) CeD PRO Symptomatic days by at least about 1, 2, 3, 4, 5, 6, or 7 days.

In embodiments, administration of IAP results in a reduction of the total GSRS score. In embodiments, administration of IAP results in a reduction of the GSRS score in one or more of the individual domains including diarrhea syndrome, indigestion syndrome, constipation syndrome, abdominal pain syndrome, and reflux syndrome. For example, administration of IAP may reduce the total GSRS score (including one or more of the individual domain scores) by at least about 20%, at least about 30%, at least about 40%, at least about 50%, at least about 60%, at least about 70%, at least about 75%, at least about 80%, at least about 85%, at least about 90%, at least about 95%, or about 100%.

In embodiments, administration of IAP results in a reduction of the number of bowel movements. In embodiments, administration of the pharmaceutical composition of the disclosure results in a reduction of the average on-treatment number of weekly bowel movements with BSFS scores of 5 to 7 (diarrhea and loose stools). For example, administration of IAP may reduce the number of bowel movements by at least about 20%, at least about 30%, at least about 40%, at least about 50%, at least about 60%, at least about 70%, at least about 75%, at least about 80%, at least about 85%, at least about 90%, at least about 95%, or about 100%. In another example, administration of IAP may reduce the number of bowel movements per week by at least about 2, at least about 3, at least about 5, at least about 10, at least about 15, or at least about 20.

In embodiments, administration of IAP results in an improvement of symptoms and well-being in the celiac disease patient as measured by the average on-treatment General Well-Being Question score.

In embodiments, administration of IAP results in an improvement of symptoms and well-being in the celiac disease patient as measured by the SF12V2 Questionnaire.

In embodiments, administration of IAP results in an improvement of symptoms and well-being in the celiac disease patient as measured by the Celiac Disease-Quality of Life Questionnaire (CeD-QoL).

In embodiments, administration of IAP results in an improvement of symptoms and well-being in the celiac disease patient as measured by the Clinician Global Assessment of Disease Activity (CGA).

In embodiments, administration of IAP treats celiac disease by modulating a non-conventional celiac disease mechanism. In embodiments, administration of IAP treats celiac disease in a manner that is orthogonal to a celiac disease mechanism. In embodiments, administration of IAP treats celiac disease in a gliadin-independent manner. In embodiments, administration of IAP treats celiac disease by improving barrier function relative to diseased state (e.g. pre-administration or without administration). In embodiments, administration of IAP treats celiac disease by increasing expression of one or more of occludin and claudin relative to diseased state (e.g. pre-administration or without administration). In embodiments, administration of IAP treats celiac disease by improving enterocyte health (e.g. viability and/or function) relative to diseased state (e.g. pre-administration or without administration). In embodiments, administration of IAP treats celiac disease by reducing non-specific inflammation (e.g. related to endotoxin, flagellin, nucleotides, etc.) relative to diseased state (e.g. pre-administration or without administration).

In accordance with embodiments of the disclosure, IAP is administered more than once daily to promote GI tight junction integrity. For example, IAP may be administered about two times daily, about three times daily, about four times daily, or about five times daily. In embodiments, the pharmaceutical composition is administered about three times daily. In embodiments, IAP is administered prior to meals, simultaneously with meals, or after meals, to reduce the effects of gluten exposure. In embodiments, IAP is administered prior to meals, such as about 15 minutes prior to meals. In embodiments, IAP is administered about 2 hours, about 90 minutes, about 60 minutes, about 55 minutes, about 45 minutes, about 40 minutes, about 35 minutes, about 30 minutes, about 25 minutes, about 20 minutes, about 15 minutes, about 10 minutes, about 9 minutes, about 8 minutes, about 7 minutes, about 6 minutes, about 5 minutes, about 4 minutes, about 3 minutes, about 2 minutes, or about 1 minute prior to meals.

In embodiments, IAP is administered after meals. In embodiments, IAP is administered about 2 hours, about 90 minutes, about 60 minutes, about 55 minutes, about 45 minutes, about 40 minutes, about 35 minutes, about 30 minutes, about 25 minutes, about 20 minutes, about 15 minutes, about 10 minutes, about 9 minutes, about 8 minutes, about 7 minutes, about 6 minutes, about 5 minutes, about 4 minutes, about 3 minutes, about 2 minutes, or about 1 minute after meals.

In embodiments, IAP may be administered for a prolonged period. Continuous IAP regimens can exhibit improving symptoms over time. For example, IAP may be administered as described herein for at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, or at least about 26 weeks. For example, IAP may be administered for at least about 8 weeks, at least about 9 weeks, at least about 10 weeks, at least about 11 weeks, or at least about 12 weeks. In embodiments, the IAP is administered for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. For example, the IAP is administered for at least about 6 months. In embodiments, the IAP may be administered for at least about 1 year, at least about 2 years, at least about 3 years, at least about 4 years, or at least about 5 years. For example, the IAP may be administered for at least about 1 year.

In embodiments, IAP compositions are administered to a subject by contacting the mucosal tissues of the gastrointestinal tract. For example, IAP may be formulated for delivery to one or more of the small intestine and large intestine. By targeting release of IAP in the affected region(s) (e.g. duodenum, jejunum and ileum, colon transversum, colon descendens, colon ascendens, colon sigmoidenum and cecum), tight junction integrity at any portion of the GI can be improved.

In embodiments, such as for patients having non-responsive or refractory celiac disease or IBS, the patient may receive adjunct therapy, which in embodiments is synergistic with IAP treatment. For example, in embodiments, the patient may receive an adjunct therapy selected from glutenases (e.g. ALV003 or latiglutenase), a peptide inhibitor of paracellular permeability (e.g. larazotide), transglutaminase inhibitors, and an antibody that targets IL-15 or IL-2/IL-15Rβ.

In embodiments, the additional therapeutic agent is an anti-inflammatory agent such as steroidal anti-inflammatory agents or non-steroidal anti-inflammatory agents (NSAIDs). Steroids, particularly the adrenal corticosteroids and their synthetic analogues, are well known in the art. Examples of corticosteroids include, without limitation, hydroxyltriamcinolone, alpha-methyl dexamethasone, beta-methyl betamethasone, beclomethasone dipropionate, clobetasol valerate, desonide, desoxymethasone, dexamethasone, diflorasone diacetate, diflucortolone valerate, fluadrenolone, fluclorolone acetonide, flumethasone pivalate, fluosinolone acetonide, fluocinonide, flucortine butylester, fluocortolone, fluprednidene (fluprednylidene) acetate, flurandrenolone, halcinonide, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, clocortelone, clescinolone, dichlorisone, difluprednate, flucloronide, flunisolide, fluoromethalone, fluperolone, fluprednisolone, hydrocortisone, meprednisone, paramethasone, prednisolone, prednisone, and budesonide. NSAIDS that may be used in the present disclosure, include but are not limited to, salicylic acid, acetyl salicylic acid, methyl salicylate, glycol salicylate, salicylmides, benzyl-2,5-diacetoxybenzoic acid, ibuprofen, fulindac, naproxen, ketoprofen, etofenamate, phenylbutazone, and indomethacin.

In embodiments, the additional therapeutic agent is an immunosuppressive agent such as azathioprine, cyclosporin, infliximab, and alemtuzumab.

In embodiments, the additional therapeutic agent is an antidiarrheal agent. Antidiarrheal agents suitable for use in the present disclosure include, but are not limited to, DPP-IV inhibitors, natural opioids, such as tincture of opium, paregoric, and codeine, synthetic opioids, such as diphenoxylate, difenoxin and loperamide, bismuth subsalicylate, lanreotide, vapreotide and octreotide, motiln antagonists, COX2 inhibitors like celecoxib, glutamine, thalidomide and traditional antidiarrheal remedies, such as kaolin, pectin, berberine and muscarinic agents.

In embodiments, the additional therapeutic agent is an antibacterial agent such as an antibiotic. Antibiotics suitable for use in the present disclosure include, but are not limited to, cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); monobactam antibiotics (aztreonam); and carbapenem antibiotics (ertapenem, doripenem, imipenem/cilastatin, and meropenem).

In embodiments, the additional therapeutic agent is a probiotic. Probiotics suitable for use in the present disclosure include, but are not limited to, Saccharomyces boulardii; Lactobacillus rhamnosus GG; Lactobacillus plantarum 299v; Clostridium butyricum M588; Clostridium difficile VP20621 (non-toxigenic C. difficile strain); combination of Lactobacillus casei, Lactobacillus acidophilus (Bio-K+CL1285); combination of Lactobacillus casei, Lactobacillus bulgaricus, Streptococcus thermophilus (Actimel); combination of Lactobacillus acidophilus, Bifidobacterium bifidum (Florajen3); combination of Lactobacillus acidophilus, Lactobacillus bulgaricus delbrueckii subsp. bulgaricus, Lactobacillus bulgaricus casei, Lactobacillus bulgaricus plantarum, Bifidobacterium longum, Bifidobacterium infantis, Bifidobacterium breve, and Streptococcus salivarius subsp. thermophilus (VSL #3).

In embodiments, the terms “patient” and “subject” are used interchangeably. In embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In embodiments, the subject and/or animal is a non-mammal, such, for example, a zebrafish.

In embodiments, methods of the disclosure are useful in treatment a human patient. In embodiments, the human is a pediatric human. In embodiments, the human is an adult human. In embodiments, the human is a geriatric human. In embodiments, the human is a female. In embodiments, the human is a male.

In embodiments, the human patient has an age in a range of from about 1 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

### Alkaline Phosphatases (APs)

The present disclosure is directed, in part, to pharmaceutical compositions, formulations, and uses of one or more alkaline phosphatases. Alkaline phosphatases are dimeric metalloenzymes that catalyze the hydrolysis of phosphate esters and dephosphorylate a variety of target substrates at physiological and higher pHs. Illustrative APs that may be utilized in the present disclosure include, but are not limited to, intestinal alkaline phosphatase (IAP; e.g., calf IAP or bovine IAP, chicken IAP, goat IAP), placental alkaline phosphatase (PLAP), placental-like alkaline phosphatase, germ cell alkaline phosphatase (GCAP), tissue non-specific alkaline phosphatase (TNAP; which is primarily found in the liver, kidney, and bone), bone alkaline phosphatase, liver alkaline phosphatase, kidney alkaline phosphatase, bacterial alkaline phosphatase, fungal alkaline phosphatase, shrimp alkaline phosphatase, modified IAP, recombinant IAP, or any polypeptide comprising alkaline phosphatase activity.

In embodiments, the present disclosure contemplates the use of mammalian alkaline phosphatases including, but are not limited to, intestinal alkaline phosphatase (IAP), placental alkaline phosphatase (PLAP), germ cell alkaline phosphatase (GCAP), and the tissue non-specific alkaline phosphatase (TNAP).

### Intestinal Alkaline Phosphatase (IAP)

In embodiments, the alkaline phosphatase is IAP. IAP is produced in the proximal small intestine and is bound to the enterocytes via a glycosyl phosphatidylinositol (GPI) anchor. Some IAP is released into the intestinal lumen in conjunction with vesicles shed by the cells and as soluble protein stripped from the cells via phospholipases. The enzyme then traverses the small and large intestine such that some active enzyme can be detected in the feces. In embodiments, the IAP is human IAP (hIAP). In embodiments, the IAP is calf IAP (cIAP), also known as bovine IAP (bIAP). There are multiple isozymes of bIAP, for example, with bIAP II and IV having higher specific activity than bIAP I. In embodiments, the IAP is any one of the cIAP or bIAP isozymes (e.g., bIAP I, II, and IV). In embodiments, the IAP is bIAP II. In embodiments, the IAP is bIAP IV.

In embodiments, the IAP of the present disclosure has greater specific enzymatic activity than commercially-available APs, e.g., calf IAP (cIAP).

### IAP Variants

Also included within the definition of IAPs are IAP variants. An IAP variant has at least one or more amino acid modifications, generally amino acid substitutions, as compared to the parental wild-type sequence. In embodiments, an IAP of the disclosure comprises an amino acid sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with any of the sequences disclosed herein. In addition, IAP variants retain most or all of their biochemical activity, measured as described herein.

### GPI Anchored Proteins

Mammalian alkaline phosphatases are GPI anchored proteins. They have signal peptides and are translated into the secretory pathway. Once in the endoplasmic reticulum (ER), the proteins are glycosylated and folded. There are two disulfide bonds as well as a single free cysteine that is apparently not accessible on the surface. In the late ER, the carboxy terminus is removed and the GPI anchor is appended. GPI anchoring is therefore a process that occurs at the carboxy terminus of the alkaline phosphatase. The inclusion of stop codons at the anchor site enables secretion of biologically active protein (presumably the homodimer). While there is no consensus sequence, the carboxy terminus includes three amino acids, termed omega, omega +1, and omega +2 which are followed by a short stretch of hydrophilic amino acids and then a stretch of hydrophobic amino acids. Without wishing to be bound by theory, it is believed that the hydrophobicity is critical for embedding the carboxy terminus in the ER membrane. There, an enzymatic reaction replaces the carboxy terminus with the GPI anchor.

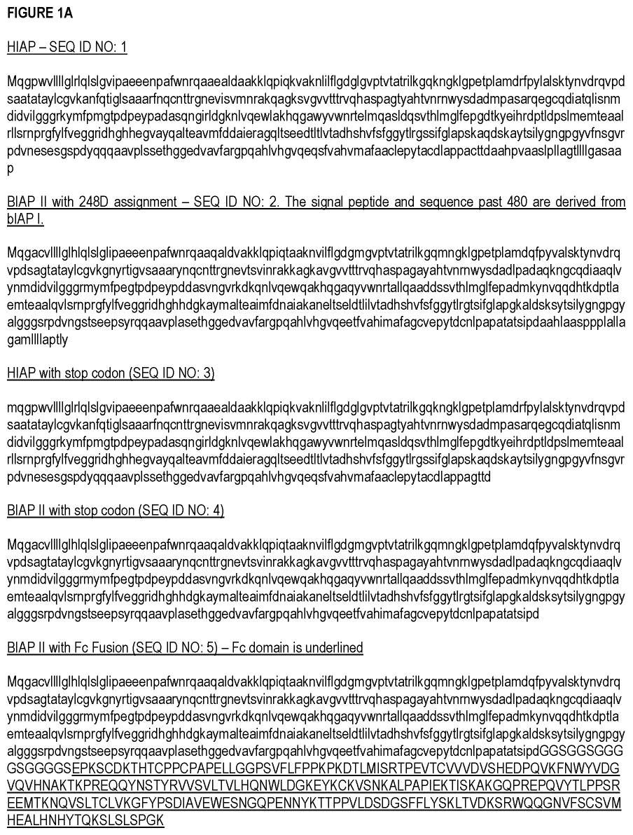

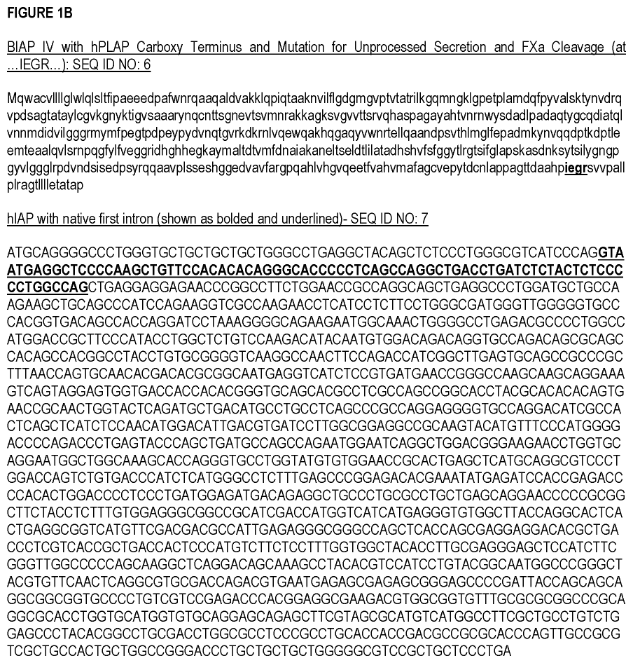

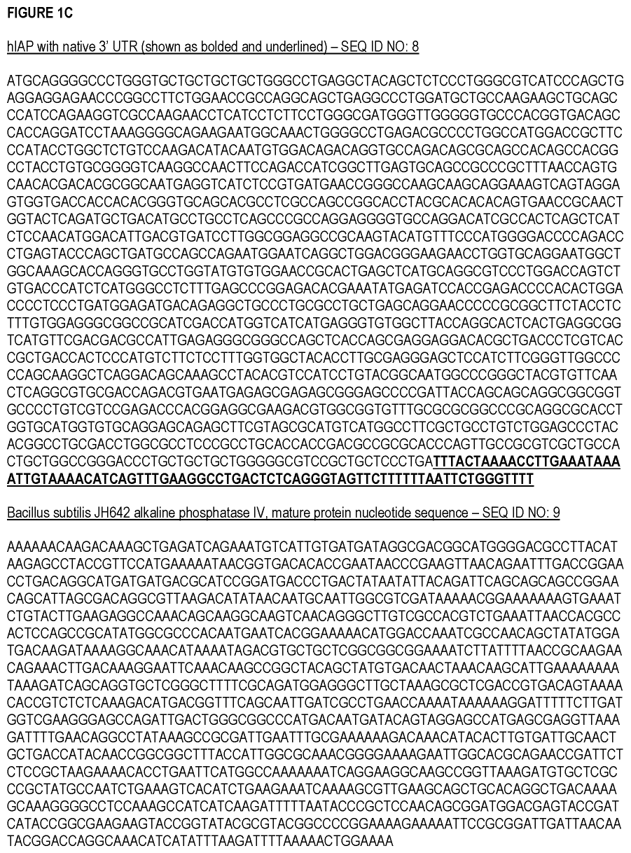

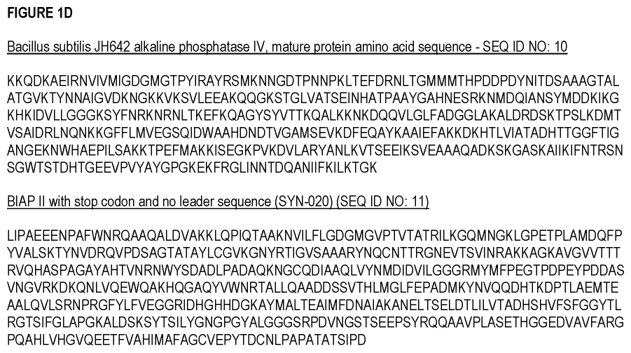

In embodiments, the IAP of the disclosure is a secreted protein; that is, in embodiments, the IAP is not GPI anchored, leading to secretion rather than intracellular retention. This can be accomplished in several ways. In embodiments, the IAP may lack the GPI anchor site, e.g. have the DAAH site removed, leading to secretion. Alternatively, this can be accomplished in embodiments, the IAP comprises a stop codon that is inserted immediately before the GPI anchor site. In embodiments, the IAP comprises a stop codon after the aspartate in the DAAH consensus site (e.g., at amino acid 503 of hIAP and bIAP IV or amino acid 506 of bIAP II). FIG. 1A-D depicts HIAP with a stop codon (SEQ ID NO: 3) and bIAP II with a stop codon (SEQ ID NO: 4).

### Human IAP

In embodiments, the IAP is human IAP (hIAP). In embodiments, the IAP is hIAP comprising the amino acid sequence of SEQ ID NO: 1 as depicted in FIG. 1A-D or a variant as described herein, as long as the hIAP variant retains at least 75, 80, 85, 90, 95, 96, 97, 98, 99 or 100% of the phosphatase activity as compared to the wild type enzyme using an assay as outlined herein.

Included within the definition of hIAP are amino acid modifications, with amino acid substitutions finding particular use in the present disclosure. For example, without wishing to be bound by theory, it is believed that a cysteine at the carboxy terminus of the AP-based agent (e.g., at position 500 of SEQ ID NO: 1) may interfere with protein folding. Accordingly, in embodiments, the AP-based agent includes a mutation of the cysteine (e.g., at position 500 of SEQ ID NO: 1). In embodiments, the cysteine is replaced with any amino acid, although glycine finds particular use in embodiments. Furthermore, the C-terminal cysteine can also be deleted.

As will be appreciated by those in the art, additional amino acid modifications can be made in hIAP as discussed herein. For example, in embodiments, a stop codon may be inserted after the aspartate in the DAAH consensus site (e.g., at amino acid 503 of hIAP). FIG. 1A-D depicts hIAP with an inserted stop codon (SEQ ID NO: 3).

### Fusion Proteins

In embodiments, the present disclosure provides for chimeric proteins. In embodiments, the present disclosure provides for chimeric fusion proteins. For example, in embodiments, the present disclosure provides an isolated or recombinant alkaline phosphatase comprising a crown domain and a catalytic domain, wherein said crown domain and said catalytic domain are obtained from different alkaline phosphatases (e.g., human and bovine alkaline phosphatases). In embodiments, the alkaline phosphatases are both human APs. In embodiments, the present disclosure provides for recombinant fusion proteins comprising human IAP and a domains of human placental alkaline phosphatases. In embodiments, the present disclosure provides for chimeric hIAP-placenta fusion proteins.

In embodiments, the AP-based agent of the disclosure is a fusion protein. In embodiments, the AP-based agent comprises an alkaline phosphatase fused to a protein domain that replaces the GPI anchor sequence. In embodiments, the alkaline phosphatase is fused to a protein domain that promotes protein folding and/or protein purification and/or protein dimerization and/or protein stability. In embodiments, the AP-based agent fusion protein has an extended serum half-life.

In embodiments, the alkaline phosphatase is fused to an immunoglobulin Fc domain and/or hinge region. In embodiments, the immunoglobulin Fc domain and/or hinge region is derived from the Fc domain and/or hinge region of an antibody (e.g., of IgG, IgA, IgD, and IgE, inclusive of subclasses (e.g. IgG1, IgG2, IgG3, and IgG4, and IgA1 and IgA2). In embodiments, the AP-based agent of the disclosure comprises an alkaline phosphatase fused to the hinge region and/or Fc domain of IgG.

In embodiments, the AP-based agent of the disclosure is a pro-enzyme. In embodiments, the activity of the proenzyme is suppressed by a carboxy terminus. In embodiments, protease removal of the carboxy terminus reactivates the enzymatic activity of the alkaline phosphatase. In embodiments, the pro-enzyme is more efficiently secreted than the enzyme without the carboxy terminus.

In embodiments, for generation of the pro-enzyme, the native carboxy terminus of the alkaline phosphatase is replaced with the analogous sequence from hPLAP. In embodiments, a mutation is made in the hydrophobic carboxy tail to promote protein secretion without cleavage of the carboxy terminus. In embodiments, a single point mutation such as a substitution of leucine with e.g., arginine is generated in the hydrophobic carboxy terminus (e.g. allpllagtl (SEQ UD NO: 12) is changed to e.g., allplragt (SEQ ID NO: 13) to result in secretion of the enzyme without removal of the carboxy terminus.

### Bovine IAPs

In embodiments, the IAP is bovine IAP (bIAP).

a. bIAP II

In embodiments, the IAP is bovine IAP II (bIAP II) or a variant as described herein, as long as the bIAP variant retains at least 75, 80, 85, 90, 95, 96, 97, 98, 99 or 100% of the phosphatase activity using an assay as outlined herein. In embodiments, the bIAP II comprises the signal peptide and carboxy terminus of bIAP I. In embodiments, the bIAP II comprises an aspartate at position 248 (similar to bIAP IV). In embodiments, the bAP II comprises the amino acid sequence of SEQ ID NO: 2. FIG. 1A-D depicts BIAP II with 248D assignment—SEQ ID NO: 2. The signal peptide and sequence past 480 are derived from bIAP I.

Also included within the definition of bIAP II are amino acid variants as described herein. For example, in embodiments, a stop codon may be inserted after the aspartate in the DAAH consensus site (e.g., at amino acid 506 of bIAP II). FIG. 1A-D depicts bIAP II with an inserted stop codon (SEQ ID NO: 4).

In embodiments, the bIAP II comprises the amino acid sequence of SEQ ID NO: 11.

BIAP II with stop codon and no leader sequence
(SYN-020) (SEQ ID NO: 11):
LIPAEEENPAFWNRQAAQALDVAKKLQPIQTAAKNVILFLGDGMGVPTV
TATRILKGQMNGKLGPETPLAMDQFPYVALSKTYNVDRQVPDSAGTATA
YLCGVKGNYRTIGVSAAARYNQCNTTRGNEVTSVINRAKKAGKAVGVVT
TTRVQHASPAGAYAHTVNRNWYSDADLPADAQKNGCQDIAAQLVYNMDI
DVILGGGRMYMFPEGTPDPEYPDDASVNGVRKDKQNLVQEWQAKHQGAQ
YVWNRTALLQAADDSSVTHLMGLFEPADMKYNVQQDHTKDPTLAEMTEA
ALQVLSRNPRGFYLFVEGGRIDHGHHDGKAYMALTEAIMFDNAIAKANE
LTSELDTLILVTADHSHVFSFGGYTLRGTSIFGLAPGKALDSKSYTSIL
YGNGPGYALGGGSRPDVNGSTSEEPSYRQQAAVPLASETHGGEDVAVFA
RGPQAHLVHGVQEETFVAHIMAFAGCVEPYTDCNLPAPATATSIPD.

### Expression Variants

In embodiments, the IAP of the disclosure is efficiently expressed and secreted from a host cell. In embodiments, the IAP of the disclosure is efficiently transcribed in a host cell. In embodiments, the IAP exhibits enhanced RNA stability and/or transport in a host cell. In embodiments, the IAP is efficiently translated in a host cell. In embodiments, the IAP exhibits enhanced protein stability.

In embodiments, the IAPs are efficiently expressed in a host cell. In embodiments, the Kozak sequence of the DNA construct encoding the AP-based agent is optimized. The Kozak sequence is the nucleotide sequence flanking the ATG start codon that instructs the ribosome to start translation. There is flexibility in the design of a Kozak sequence, but one canonical sequence is GCCGCCACCATGG (SEQ ID NO: 14). The purine in the −3 position and the G in the +4 position are the most important bases for translation initiation. For hIAP, bIAP II, and bIAP IV, the second amino acid, that is, the one after the initiator methionine, is glutamine. Codons for glutamine all have a C in the first position. Thus, their Kozak sequences all have an ATGC sequence. Accordingly, in embodiments, the ATGC sequence is changed to ATGG. This can be achieved by changing the second amino acid to a glycine, alanine, valine, aspartate, or glutamic acid, all of whose codons have a G in the first position. These amino acids may be compatible with signal peptide function. In embodiments, the entire signal peptide is substituted for peptide having a canonical Kozak sequence and is derived from a highly expressed protein such as an immunoglobulin.

In embodiments, the signal peptide of the IAP may be deleted and/or substituted. For example, the signal peptide may be deleted, mutated, and/or substituted (e.g., with another signal peptide) to ensure optimal protein expression.

In embodiments, the DNA construct encoding the IAP of the disclosure comprises untranslated DNA sequences. Such sequences include an intron, which may be heterologous to the IAP protein or native to the IAP protein including the native first and/or second intron and/or a native 3′ UTR. Without wishing to be bound by theory, it is believed that include of these sequences enhance protein expression by stabilizing the mRNA. Accordingly, in embodiments, the DNA construct encoding the IAP of the disclosure comprises the 5′UTR and/or the 3′UTR. Provided in FIG. 1A-D are illustrative IAP DNA sequences with a first intron and a 3′UTR, including hIAP with native first intron (shown as bolded and underlined)—SEQ ID NO: 7; and hIAP with native 3′ UTR (shown as bolded and underlined)—SEQ ID NO: 8.

In embodiments, the IAP of the disclosure comprises a nucleotide sequence having at least about 60% (e.g. about or at least about 60%, or about or at least about 61%, or about or at least about 62%, or about or at least about 63%, or about or at least about 64%, or about or at least about 65%, or about or at least about 66%, or about or at least about 67%, or about or at least about 68%, or about or at least about 69%, or about or at least about 70%, or about or at least about 71%, or about or at least about 72%, or about or at least about 73%, or about or at least about 74%, or about or at least about 75%, or about or at least about 76%, or about or at least about 77%, or about or at least about 78%, or about or at least about 79%, or about or at least about 80%, or about or at least about 81%, or about or at least about 82%, or about or at least about 83%, or about or at least about 84%, or about or at least about 85%, or about or at least about 86%, or about or at least about 87%, or about or at least about 88%, or about or at least about 89%, or about or at least about 90%, or about or at least about 91%, or about or at least about 92%, or about or at least about 93%, or about or at least about 94%, or about or at least about 95%, or about or at least about 96%, or about or at least about 97%, or about or at least about 98%, or about or at least about 99%) sequence identity with any of the sequences disclosed herein.

In embodiments, the IAP of the disclosure may comprise an amino acid sequence having one or more amino acid mutations relative to any of the protein sequences described herein. In embodiments, the one or more amino acid mutations may be independently selected from substitutions, insertions, deletions, and truncations. In embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β-methyl amino acids, C α-methyl amino acids, N α-methyl amino acids, and amino acid analogs in general).

Mutations may be made to the IAP of the disclosure to select for agents with desired characteristics. For examples, mutations may be made to generate IAPs with enhanced catalytic activity or protein stability. In embodiments, directed evolution may be utilized to generate IAPs of the disclosure. For example, error-prone PCR and DNA shuffling may be used to identify mutations in the bacterial alkaline phosphatases that confer enhanced activity.

### Methods of Making IAP of the Disclosure

The IAPs of the disclosure are made using standard molecular biology techniques. For example, nucleic acid compositions encoding the IAPs of the disclosure are also provided, as well as expression vectors containing the nucleic acids and host cells transformed with the nucleic acid and/or expression vector compositions. As will be appreciated by those in the art, the protein sequences depicted herein can be encoded by any number of possible nucleic acid sequences, due to the degeneracy of the genetic code.

As is known in the art, the nucleic acids encoding the components of the disclosure can be incorporated into expression vectors as is known in the art, and depending on the host cells, used to produce the IAP compositions of the disclosure. Generally, the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors of the disclosure are then transformed into any number of different types of host cells as is well known in the art, including mammalian, bacterial, yeast, insect and/or fungal cells, with mammalian cells (e.g. CHO cells), finding use in many embodiments.

The IAPs of the disclosure are made by culturing host cells comprising the expression vector(s) as is well known in the art. Once produced, traditional purification steps are done.

### Formulations

The present disclosure provides the described IAP (and/or additional therapeutic agents) in various formulations. Any IAP (and/or additional therapeutic agents) described herein can take the form of tablets, pills, pellets, capsules, capsules containing liquids, capsules containing multiparticulates, powders, solutions, emulsion, drops, suppositories, emulsions, aerosols, sprays, suspensions, delayed-release formulations, sustained-release formulations, controlled-release formulations, or any other form suitable for use.

The formulations comprising the IAP (and/or additional therapeutic agents) may conveniently be presented in unit dosage forms. For example, the dosage forms may be prepared by methods which include the step of bringing the therapeutic agents into association with a carrier, which constitutes one or more accessory ingredients. For example, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by press tableting).

In embodiments, the IAP (and/or additional therapeutic agents) described herein are formulated as compositions adapted for a mode of administration described herein.

In embodiments, the IAP and an additional therapeutic agent are co-formulated.

In embodiments, the formulation comprising IAP is resistant to compression and therefore suitable for tableting. In embodiments, the formulation comprising IAP is resistant to compression and therefore suitable for tableting. The IAP can be provided in a powder form that is then tableted, e.g., by physical compression of dried materials.

In embodiments, the IAP of the disclosure is stable and/or active in the GI tract, e.g. in one or more of the mouth, esophagus, stomach, duodenum, small intestine, duodenum, jejunum, ileum, large intestine, colon transversum, colon descendens, colon ascendens, colon sigmoidenum, cecum, and rectum. In embodiments, the IAP is stable in the large intestine, optionally selected from one or more of colon transversum, colon descendens, colon ascendens, colon sigmoidenum and cecum. In embodiments, the IAP is stable in the small intestine, optionally selected from one or more of duodenum, jejunum, and ileum. In embodiments, the IAP is resistant to proteases in the GI tract, including for example, the small intestine. In embodiments, the IAP is substantially active at a pH of about 5.0 or above. For example, the IAP may be substantially active at a pH of about 4.0 to about 12, e.g. about 4.0, or about 4.1, or about 4.2, or about 4.3, or about 4.4, or about 4.5, or about 4.6, or about 4.7, or about 4.8, or about 4.9, or about 5.0, or about 5.1, or about 5.2, or about 5.3, or about 5.4, or about 5.5, or about 5.6, or about 5.7, or about 5.8, or about 5.9, or about 6.0, or about 6.1, or about 6.2, or about 6.3, or about 6.4, or about 6.5, or about 6.6, or about 6.7, or about 6.8, or about 6.9, or about 7.0, or about 7.1, or about 7.2, or about 7.3, or about 7.4, or about 7.5, or about 8.0, or about 8.5, or about 9.0, or about 9.5, or about 10.0, or about 10.5, or about 11.0, or about 11.5, or about 12.0 (including, for example, via formulation, as described herein). In embodiments, stable refers to an enzyme that has a long enough half-life and maintains sufficient activity for therapeutic effectiveness.

In embodiments, the IAP of the disclosure is stable in chyme. In order to assess IAP stability in chyme, samples of IAPs are incubated in human chyme at 37° C. Stability is then evaluated by assessing aliquots withdrawn from the incubated samples at 0, 0.5, 1, 2, 3, 4, 5, and 6 hours for AP activity using a para-nitrophenyl phosphate (pNPP) AP substrate. Different chyme specimens can be used for evaluation of stability, including mixed chyme samples. Chyme samples are characterized for pH, liquid content, and protease activity.

In embodiments, the IAP described herein includes derivatives that are modified, i.e., by the covalent attachment of any type of molecule to the alkaline phosphatase such that covalent attachment does not prevent the activity of the enzyme. For example, but not by way of limitation, derivatives include alkaline phosphatases that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out, including, but not limited to specific chemical cleavage, acetylation, formylation, metabolic synthesis of tunicamycin, etc. Additionally, the derivative can contain one or more non-classical amino acids. In embodiments, the IAP is glycosylated to ensure proper protein folding.

### Pharmaceutically Acceptable Salts

The IAP described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

The term “pharmaceutically acceptable salt” also refers to a salt of the alkaline phosphatases having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

In embodiments, the compositions described herein are in the form of pharmaceutically acceptable salts. In embodiments, the formulation comprises 5%, about 10%, about 15%, about 20%, about 25%, about 30%, about 31%, about 32%, about 33%, about 34%, about 35%, about 36%, about 37%, about 38%, about 39%, about 40%, about 41%, about 42%, about 43%, about 43%, about 44%, about 45%, about 46%, about 47%, about 48%, about 49%, or about 50% by weight pharmaceutically acceptable salts.

### Pharmaceutical Excipients

Further, any IAP described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, cellulose, hypromellose, lactose, sucrose, trehalose, malt, rice, flour, chalk, silica gel, sodium stearate, glycerol monostearate, talc, sodium chloride, dried skim milk, glycerol, propylene, glycol, povidone, crosspovidone, water, ethanol and the like. Any agent described herein, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

A suitable pharmaceutical excipient for the purposes of tableting can be Ludipress (Lactose, povidone, crospovidone; CAS-No.: 5989-81-1+9003-39-8).

Where necessary, the IAP pharmaceutical compositions (and/or additional therapeutic agents) can include a solubilizing agent. Also, the agents can be delivered with a suitable vehicle or delivery device. Combination therapies outlined herein can be co-delivered in a single delivery vehicle or delivery device.

In embodiments, the IAP (and/or additional therapeutic agents) described herein are formulated as compositions adapted for oral administration. Compositions for oral delivery can be in the form of tablets, lozenges, aqueous or oily suspensions, granules, powders, sprinkles, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration to provide a sustained action over an extended period of time. Selectively permeable membranes surrounding an osmotically active agent driving any IAP (and/or additional therapeutic agents) described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include excipients such as mannitol, lactose, starch, magnesium stearate, sodium saccharin, cellulose, ethacrylic acid and derivative polymers thereof, and magnesium carbonate. In embodiments, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

In embodiments, the IAP (and/or additional therapeutic agent) are formulated as solid dosage forms such as tablets, dispersible powders, granules, and capsules. In embodiments, the IAP (and/or additional therapeutic agent) is formulated as a capsule. In embodiments, the IAP (and/or additional therapeutic agent) is formulated as a tablet. In embodiments, the (and/or additional therapeutic agent) is formulated as a soft-gel capsule. In embodiments, the IAP (and/or additional therapeutic agent) is formulated as a gelatin capsule.

In embodiments, the formulations of the IAP may additionally comprise a pharmaceutically acceptable carrier or excipient. As one skilled in the art will recognize, the formulations can be in any suitable form appropriate for the desired use and route of administration.

In some dosage forms, the agents described herein are mixed with at least one inert, pharmaceutically acceptable excipient or carrier such as sodium citrate, dicalcium phosphate, etc., and/or a) fillers or extenders such as starches, lactose, sucrose, glucose, mannitol, silicic acid, microcrystalline cellulose, and Bakers Special Sugar, etc., b) binders such as, for example, carboxymethylcellulose, alginates, gelatin, polyvinylpyrrolidone, sucrose, acacia, polyvinyl alcohol, polyvinylpyrrolidone, methylcellulose, hydroxypropyl cellulose (HPC), and hydroxymethyl cellulose etc., c) humectants such as glycerol, etc., d) disintegrating agents such as agar-agar, calcium carbonate, potato or tapioca starch, alginic acid, certain silicates, sodium carbonate, cross-linked polymers such as crospovidone (cross-linked polyvinylpyrrolidone), croscarmellose sodium (cross-linked sodium carboxymethylcellulose), sodium starch glycolate, etc., e) solution retarding agents such as paraffin, etc., f) absorption accelerators such as quaternary ammonium compounds, etc., g) wetting agents such as, for example, cetyl alcohol and glycerol monostearate, etc., h) absorbents such as kaolin and bentonite clay, etc., and i) lubricants such as talc, calcium stearate, magnesium stearate, solid polyethylene glycols, sodium lauryl sulfate, glyceryl behenate, etc., and mixtures of such excipients. One of skill in the art will recognize that particular excipients may have two or more functions in the oral dosage form. In the case of an oral dosage form, for example, a capsule or a tablet, the dosage form may also comprise buffering agents.

### Surface Active Agents

The formulation can additionally include a surface active agent. Surface active agents suitable for use in the present disclosure include, but are not limited to, any pharmaceutically acceptable, non-toxic surfactant. Classes of surfactants suitable for use in the compositions of the disclosure include, but are not limited to polyethoxylated fatty acids, PEG-fatty acid diesters, PEG-fatty acid mono- and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-olyoxypropylene block copolymers, sorbitan fatty acid esters, lower alcohol fatty acid esters, ionic surfactants, and mixtures thereof. In embodiments, compositions of the disclosure may comprise one or more surfactants including, but not limited to, sodium lauryl sulfate, polysorbate 20, polysorbate 40, polysorbate 60, polysorbate 80, and triethyl citrate.

The formulation can also contain pharmaceutically acceptable plasticizers to obtain the desired mechanical properties such as flexibility and hardness. Such plasticizers include, but are not limited to, triacetin, citric acid esters, triethyl citrate, phthalic acid esters, dibutyl sebacate, cetyl alcohol, polyethylene glycols, polysorbates or other plasticizers.

The formulation can also include one or more application solvents. Some of the more common solvents that can be used to apply, for example, a delayed-release coating composition include isopropyl alcohol, acetone, methylene chloride and the like.

The formulation can also include one or more alkaline materials. Alkaline material suitable for use in compositions of the disclosure include, but are not limited to, sodium, potassium, calcium, magnesium and aluminum salts of acids such as phosphoric acid, carbonic acid, citric acid and other aluminum/magnesium compounds. In addition, the alkaline material may be selected from antacid materials such as aluminum hydroxides, calcium hydroxides, magnesium hydroxides and magnesium oxide.

In embodiments, the formulation can additionally include magnesium and/or zinc. Without wishing to be bound by theory, the inclusion of magnesium and/or zinc in the formulation promotes protein folding (e.g., dimer formation) and bioactivity of the IAP. In embodiments, the formulation can include magnesium at a concentration of from about 1 μM to greater than 5 mM (e.g., from about 1 μM to more than 5 mM), inclusive of all ranges and values therebetween. In embodiments, the magnesium is present in the formulation at 1.0 mM. In embodiments, the formulation can include zinc at a concentration of about 1 μM to greater than 1 mM (e.g., from about 1 μM to more than 1 mM), inclusive of all ranges and values therebetween. In embodiments, the zinc is present in the formulation at 0.1 mM. In embodiments, the formulation of the present disclosure is substantially free of metal chelators.

In embodiments, the pH of the formulation ensures that the IAP is properly folded (e.g., dimer formation) and is bioactive. In embodiments, the formulation is maintained at a pH such that the amino acids which coordinate the binding of magnesium and/or zinc within the AP-based agent are not protonated. Protonation of such coordinating amino acids may lead to loss of metal ions and bioactivity and dimer disassociation. In embodiments, the pH of the formulation is greater than about 6, about 6.5, about 7, about 7.5, about 8, about 8.5, about 9, about 9.5, about 10, about 10.5, about 11, about 11.5, or about 12.

Besides inert diluents, the oral compositions can also include adjuvants such as sweetening, flavoring, and perfuming agents.

### Delivery

Various methods may be used to formulate and/or deliver the agents described herein to a location of interest. For example, the IAP (and/or additional therapeutic agents) described herein may be formulated for delivery to the GI tract. The GI tract includes organs of the digestive system such as mouth, esophagus, stomach, duodenum, small intestine, large intestine and rectum and includes all subsections thereof (e.g. the small intestine may include the duodenum, jejunum and ileum; the large intestine may include the colon transversum, colon descendens, colon ascendens, colon sigmoidenum and cecum). For example, the IAP (and/or additional therapeutic agents) described herein may be formulated for delivery to one or more of the stomach, small intestine, large intestine and rectum and includes all subsections thereof (e.g. duodenum, jejunum and ileum, colon transversum, colon descendens, colon ascendens, colon sigmoidenum and cecum). In embodiments, the compositions described herein may be formulated to deliver to the gut. In embodiments, the compositions described herein may be formulated to deliver to the upper or lower GI tract. In embodiments, the IAP (and/or additional therapeutic agents) may be administered to a subject, by, for example, directly or indirectly contacting the mucosal tissues of the GI tract.

In embodiments, the administration of the IAP (and/or additional therapeutic agents) is into the GI tract via, for example, oral delivery, nasogastral tube, intestinal intubation (e.g. an enteral tube or feeding tube such as, for example, a jejunal tube or gastro-jejunal tube, etc.), direct infusion (e.g., duodenal infusion), endoscopy, colonoscopy, sigmoidoscopy or enema.

For example, in embodiments, the present disclosure provides modified release formulations comprising at least one IAP (and/or additional therapeutic agents), wherein the formulation releases a substantial amount of the IAP (and/or additional therapeutic agents) into one or more regions of the GI tract. For example, the formulation may release at least about 60% of the AP-based agent after the stomach and into one or more regions of the GI tract.

In embodiments, the modified-release formulation of the present disclosure releases at least 60% of the IAP (or additional therapeutic agents) after the stomach into one or more regions of the intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the IAP (or additional therapeutic agents) in the intestines.

In embodiments, the modified-release formulation of the present disclosure releases at least 60% of the IAP (or additional therapeutic agents) in the small intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the AP-based agent (or additional therapeutic agents) in the small intestine (e.g., one or more of duodenum, jejunum, ileum, and ileocecal junction).

In embodiments, the modified-release formulation of the present disclosure releases at least 60% of the IAP (or additional therapeutic agents) in the large intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the AP-based agent (or additional therapeutic agents) in the large intestine (e.g., one or more of cecum, ascending, transverse, descending or sigmoid portions of the colon, and rectum).

In embodiments, the modified-release formulation does not substantially release the AP-based agent (or additional therapeutic agents) in the stomach.

In embodiments, the modified-release formulation releases the IAP (or additional therapeutic agents) above a specific pH. For example, in embodiments, the modified-release formulation is substantially stable in an acidic environment and substantially unstable (e.g., dissolves rapidly or is physically unstable) in a near neutral to alkaline environment. In embodiments, stability is indicative of not substantially releasing while instability is indicative of substantially releasing. For example, in embodiments, the modified-release formulation is substantially stable at a pH of about 7.0 or less, or about 6.5 or less, or about 6.0 or less, or about 5.5 or less, or about 5.0 or less, or about 4.5 or less, or about 4.0 or less, or about 3.5 or less, or about 3.0 or less, or about 2.5 or less, or about 2.0 or less, or about 1.5 or less, or about 1.0 or less. In embodiments, the present formulations are stable in lower pH areas and therefore do not substantially release in, for example, the stomach. In embodiments, the modified-release formulation is substantially stable at a pH of about 1 to about 5 or lower and substantially unstable at pH values that are greater. In embodiments, the modified-release formulation does not substantially release in the stomach. In embodiments, the modified-release formulation substantially releases in the small intestine (e.g. one or more of the duodenum, jejunum, and ileum) and/or large intestine (e.g. one or more of the cecum, ascending colon, transverse colon, descending colon, and sigmoid colon). In embodiments, modified-release formulation is substantially stable at a pH of about 4 to about 7 or lower and consequentially is substantially unstable at pH values that are greater and therefore is not substantially released in the stomach and/or proximal small intestine (e.g. one or more of the duodenum, jejunum). In embodiments, the modified-release formulation substantially releases in the distal small intestine or large intestine (e.g. one or more of the cecum, ascending colon, transverse colon, descending colon, and sigmoid colon). In embodiments, the pH values recited herein may be adjusted as known in the art to account for the state of the subject, e.g. whether in a fasting or postprandial state.

In embodiments, the modified-release formulation is substantially stable in gastric fluid and substantially unstable in intestinal fluid and, accordingly, is substantially released in the small intestine (e.g. one or more of the duodenum, jejunum, and ileum) and/or large intestine (e.g. one or more of the cecum, ascending colon, transverse colon, descending colon, and sigmoid colon).

In embodiments, the modified-release formulation is stable in gastric fluid or stable in acidic environments. These modified-release formulations release about 30% or less by weight of the alkaline phosphatase and/or additional therapeutic agent in the modified-release formulation in gastric fluid with a pH of about 4 to about 5 or less, or simulated gastric fluid with a pH of about 4 to about 5 or less, in about 15, or about 30, or about 45, or about 60, or about 90 minutes. Modified-release formulations of the of the disclosure may release from about 0% to about 30%, from about 0% to about 25%, from about 0% to about 20%, from about 0% to about 15%, from about 0% to about 10%, about 5% to about 30%, from about 5% to about 25%, from about 5% to about 20%, from about 5% to about 15%, from about 5% to about 10% by weight of the alkaline phosphatase and/or additional therapeutic agent in the modified-release formulation in gastric fluid with a pH of 4-5, or less or simulated gastric fluid with a pH of 4-5 or less, in about 15, or about 30, or about 45, or about 60, or about 90 minutes. Modified-release formulations of the disclosure may release about 1%, about 2%, about 3%, about 4%, about 5%, about 6%, about 7%, about 8%, about 9%, or about 10% by weight of the total alkaline phosphatase and/or additional therapeutic agent in the modified-release formulation in gastric fluid with a pH of 5 or less, or simulated gastric fluid with a pH of 5 or less, in about 15, or about 30, or about 45, or about 60, or about 90 minutes.

In embodiments, the modified-release formulation is unstable in intestinal fluid. These modified-release formulations release about 70% or more by weight of the alkaline phosphatase and/or additional therapeutic agent in the modified-release formulation in intestinal fluid or simulated intestinal fluid in about 15, or about 30, or about 45, or about 60, or about 90 minutes. In embodiments, the modified-release formulation is unstable in near neutral to alkaline environments. These modified-release formulations release about 70% or more by weight of the alkaline phosphatase and/or additional therapeutic agent in the modified-release formulation in intestinal fluid with a pH of about 4-5 or greater, or simulated intestinal fluid with a pH of about 4-5 or greater, in about 15, or about 30, or about 45, or about 60, or about 90 minutes. A modified-release formulation that is unstable in near neutral or alkaline environments may release 70% or more by weight of alkaline phosphatase and/or additional therapeutic agent in the modified-release formulation in a fluid having a pH greater than about 5 (e.g., a fluid having a pH of from about 5 to about 14, from about 6 to about 14, from about 7 to about 14, from about 8 to about 14, from about 9 to about 14, from about 10 to about 14, or from about 11 to about 14) in from about 5 minutes to about 90 minutes, or from about 10 minutes to about 90 minutes, or from about 15 minutes to about 90 minutes, or from about 20 minutes to about 90 minutes, or from about 25 minutes to about 90 minutes, or from about 30 minutes to about 90 minutes, or from about 5 minutes to about 60 minutes, or from about 10 minutes to about 60 minutes, or from about 15 minutes to about 60 minutes, or from about 20 minutes to about 60 minutes, or from about 25 minutes to about 90 minutes, or from about 30 minutes to about 60 minutes.

Examples of simulated gastric fluid and simulated intestinal fluid include, but are not limited to, those disclosed in the 2005 Pharmacopeia 23NF/28USP in Test Solutions at page 2858 and/or other simulated gastric fluids and simulated intestinal fluids known to those of skill in the art, for example, simulated gastric fluid and/or intestinal fluid prepared without enzymes.

In embodiments, the modified-release formulation of the disclosure is substantially stable in chyme. For example, there is, in embodiments, a loss of less than about 50% or about 40%, or about 30%, or about 20%, or about 10% of AP-based agent activity in about 10, or 9, or 8, or 7, or 6, or 5, or 4, or 3, or 2, or 1 hour from administration.

In embodiments, the modified-release formulations of the present disclosure are designed for immediate release (e.g. upon ingestion). In embodiments, the modified-release formulations may have sustained-release profiles, i.e. slow release of the active ingredient(s) in the body (e.g., GI tract) over an extended period of time. In embodiments, the modified-release formulations may have a delayed-release profile, i.e. not immediately release the active ingredient(s) upon ingestion; rather, postponement of the release of the active ingredient(s) until the composition is lower in the GI tract; for example, for release in the small intestine (e.g., one or more of duodenum, jejunum, ileum) or the large intestine (e.g., one or more of cecum, ascending, transverse, descending or sigmoid portions of the colon, and rectum). For example, a composition can be enteric-coated to delay release of the active ingredient(s) until it reaches the small intestine or large intestine.

### Enteric Coating

In embodiments, the formulations of the present disclosure (e.g. IAP as a powder or tablet) are coated to provide protection of the active agent in the GI tract, including the stomach. For example, in embodiments, the present formulations can be encapsulated in an enterically-coated capsule. Additionally, in embodiments, the formulations (e.g. IAP as a powder or tablet) itself is coated with one or more coatings, e.g. one or more modified-release coatings as described herein (e.g. after a step of granulating the powder). Further, in embodiments, the present powder formulations (e.g. AP-based agent as a powder) can be compressed into a tablet that is enterically coated.

In embodiments, the modified-release formulation of the present disclosure may utilize one or more modified-release coatings such as delayed-release coatings to provide for effective, delayed yet substantial delivery of the alkaline phosphatase to the GI tract together with, optionally, additional therapeutic agents.

In embodiments, the modified-release formulation of the present disclosure may utilize one or more modified-release coatings such as delayed-release coatings to provide for effective, delayed yet substantial delivery of the IAP to the intestines together with, optionally, other additional therapeutic agents.

In embodiments, the delayed-release coating includes an enteric agent that is substantially stable in acidic environments and substantially unstable in near neutral to alkaline environments. In embodiments, the delayed-release coating contains an enteric agent that is substantially stable in gastric fluid. The enteric agent can be selected from, for example, solutions or dispersions of methacrylic acid copolymers, cellulose acetate phthalate, hydroxypropylmethyl cellulose phthalate, polyvinyl acetate phthalate, carboxymethylethylcellulose, and EUDRAGIT®-type polymer (poly(methacrylic acid, methylmethacrylate), hydroxypropyl methylcellulose acetate succinate, cellulose acetate trimellitate, shellac or other suitable enteric coating polymers. The polymers are described in international pharmacopeias such as Ph. Eur., USP/NF, DMF, and JPE. The EUDRAGIT®-type polymers include, for example, EUDRAGIT® FS 30D, L 30 D-55, L 100-55, L 100, L 12, 5, L 12, 5 P, RL 30 D, RL PO, RL 100, RL 12, 5, RS 30 D, RS PO, RS 100, RS 12, 5, NE 30 D, NE 40 D, NM 30 D, S 100, S 12, 5, and S 12, 5 P. Similar polymers include Kollicoat® MAE 30 DP and Kollicoat® MAE 100 P. In embodiments, one or more of EUDRAGIT® FS 30D, L 30 D-55, L 100-55, L 100, L 12, 5, L 12, 5 P RL 30 D, RL PO, RL 100, RL 12, 5, RS 30 D, RS PO, RS 100, RS 12, 5, NE 30 D, NE 40 D, NM 30 D, S 100, S 12, 5 S 12, 5 P, Kollicoat® MAE 30 DP and Kollicoat® MAE 100 P is used. In embodiments, the enteric agent may be a combination of the foregoing solutions or dispersions. In embodiments, the delayed-release coating includes the enteric agent EUDRAGIT® L 100.

By way of non-limiting example, there are various EUDRAGIT formulations that dissolve at rising pH, with formulations that dissolve at pH>5.5 (EUDRAGIT L30 D-550), pH>6.0 (EUDRAGIT L12, 5), and pH>7.0 (EUDRAGIT FS 30D). Since the ileum has the highest pH in the small intestine, ranging from 7.3 to 7.8, the use of EUDRAGIT FS 30D as an enteric agent, may delay dissolution until the ileum thereby localizing the release of the AP-based agent to the ileum. However, the jejunum has a pH that can range from 6.6 to 7.4, therefore, various EUDRAGIT formulations can be used to target release to this segment of the intestine. The different types of EUDRAGIT can be combined with each other, or multiple different types of EUDRAGIT coatings can be combined to fine tune the dissolution profile to achieve targeted delivery to achieve optimal function. For example, EUDRAGIT L100, EUDRAGIT S100, and triethyl citrate may be mixed together at a ratio of, for example, about 72.7/18.2/9.1, to form a coating that substantially releases at a pH of greater than about 6.2. In another example, EUDRAGIT L100, EUDRAGIT S100, and triethyl citrate may be mixed together at a ratio of, for example, about 30/60.9/9, to form a coating that substantially releases at a pH of greater than about 6.7. In a further example, DuoCoat™ (Kuecept, Ltd.) may be used that uses two coatings of enteric polymers (like EUDRAGIT), an outer layer, and an inner layer of partially neutralized enteric polymer and a buffer agent. The DuoCoat™ technology allows more rapid release of the therapeutic agent initiated at the targeted pH compared to a single coating of the enteric polymer (Liu et al., 2010, European J. Pharmaceutics and Biopharmaceuticals 47:311, the entire contents of all of which are incorporated herein by reference). Release was demonstrated to be targeted to the ileum and/or ileoceacal junction in 10 healthy volunteers (Varum et al., 2013, European J. Pharmaceutics and Biopharmaceuticals 84:573, the entire contents of all of which are incorporated herein by reference).

In embodiments, one or more coating system additives are used with the enteric agent. For example, one or more PlasACRYL™ additives may be used as an anti-tacking agent coating additive. Illustrative PlasACRYL™ additives include, but are not limited to PlasACRYL™ HTP20 and PlasACRYL™ T20.

In embodiments, the delayed-release coating may degrade as a function of time when in aqueous solution without regard to the pH and/or presence of enzymes in the solution. Such a coating may comprise a water insoluble polymer. Its solubility in aqueous solution is therefore independent of the pH. The term “pH independent” as used herein means that the water permeability of the polymer and its ability to release pharmaceutical ingredients is not a function of pH and/or is only very slightly dependent on pH. Such coatings may be used to prepare, for example, sustained release formulations. Suitable water insoluble polymers include pharmaceutically acceptable non-toxic polymers that are substantially insoluble in aqueous media, e.g., water, independent of the pH of the solution. Suitable polymers include, but are not limited to, cellulose ethers, cellulose esters, or cellulose ether-esters, i.e., a cellulose derivative in which some of the hydroxy groups on the cellulose skeleton are substituted with alkyl groups and some are modified with alkanoyl groups. Examples include ethyl cellulose, acetyl cellulose, nitrocellulose, and the like. Other examples of insoluble polymers include, but are not limited to, lacquer, and acrylic and/or methacrylic ester polymers, polymers or copolymers of acrylate or methacrylate having a low quaternary ammonium content, or mixture thereof and the like. Other examples of insoluble polymers include EUDRAGIT RS®, EUDRAGIT RL®, and EUDRAGIT NE®. Insoluble polymers useful in the present disclosure include polyvinyl esters, polyvinyl acetals, polyacrylic acid esters, butadiene styrene copolymers, and the like. In embodiments, colonic delivery is achieved by use of a slowly-eroding wax plug (e.g., various PEGS, including for example, PEG6000) or pectin. In embodiments, the present disclosure contemplates the use of a delayed-release coating that degrade as a function of time which comprises a swell layer comprising croscarmellos sodium and hydroxyproplycellulose. In embodiments, the formulation may further include an osmotic rupture coating that comprises ethylcellulose such as ethylcellulose dispersions.

Alternatively, the stability of the modified-release formulation can be enzyme-dependent. Delayed-release coatings that are enzyme dependent will be substantially stable in fluid that does not contain a particular enzyme and substantially unstable in fluid containing the enzyme. The delayed-release coating will essentially disintegrate or dissolve in fluid containing the appropriate enzyme. Enzyme-dependent control can be brought about, for example, by using materials which release the active ingredient only on exposure to enzymes in the intestine, such as galactomannans. Also, the stability of the modified-release formulation can be dependent on enzyme stability in the presence of a microbial enzyme present in the gut flora. For example, in embodiments, the delayed-release coating may be degraded by a microbial enzyme present in the gut flora. In embodiments, the delayed-release coating may be degraded by bacteria present in the small intestine. In embodiments, the delayed-release coating may be degraded by bacteria present in the large intestine.

In embodiments, the modified release formulation is designed for release in the colon. Various colon-specific delivery approaches may be utilized. For example, the modified release formulation may be formulated using a colon-specific drug delivery system (CODES) as described for example, in Li et al., AAPS PharmSciTech (2002), 3(4): 1-9, the entire contents of which are incorporated herein by reference. Drug release in such a system is triggered by colonic microflora coupled with pH-sensitive polymer coatings. For example, the formulation may be designed as a core tablet with three layers of polymer. The first coating is an acid-soluble polymer (e.g., EUDRAGIT E), the outer coating is enteric, along with a hydroxypropyl methylcellulose barrier layer interposed in between. In embodiments, colon delivery may be achieved by formulating the alkaline phosphatase (and/or additional therapeutic agent) with specific polymers that degrade in the colon such as, for example, pectin. The pectin may be further gelled or crosslinked with a cation such as a zinc cation. In embodiments, the formulation is in the form of ionically crosslinked pectin beads which are further coated with a polymer (e.g., EUDRAGIT polymer). Additional colon specific formulations include, but are not limited to, pressure-controlled drug delivery systems (prepared with, for example, ethylcellulose) and osmotic controlled drug delivery systems (i.e., ORDS-CT).

Formulations for colon specific delivery of the IAP (and/or additional therapeutic agents), as described herein, may be evaluated using, for example, in vitro dissolution tests. For example, parallel dissolution studies in different buffers may be undertaken to characterize the behavior of the formulations at different PH levels. Alternatively, in vitro enzymatic tests may be carried out. For example, the formulations may be incubated in fermenters containing suitable medium for bacteria, and the amount of drug released at different time intervals is determined. Drug release studies can also be done in buffer medium containing enzymes or rat or guinea pig or rabbit cecal contents and the amount of drug released in a particular time is determined. In embodiments, in vivo evaluations may be carried out using animal models such as dogs, guinea pigs, rats, and pigs. Further, clinical evaluation of colon specific drug delivery formulations may be evaluated by calculating drug delivery index (DDI) which considers the relative ratio of RCE (relative colonic tissue exposure to the drug) to RSC (relative amount of drug in blood i.e. that is relative systemic exposure to the drug). Higher drug DDI indicates better colon drug delivery. Absorption of drugs from the colon may be monitored by colonoscopy and intubation.

In embodiments, the present formulations provide for substantial uniform dissolution of the AP-based agent (and/or additional therapeutic agent) in the area of release in the GI tract. In embodiments, the present formulation minimizes patchy or heterogeneous release of the AP-based agent.

In embodiments, the present disclosure provides for modified-release formulations that release multiple doses of the AP-based agent, at different locations along the intestines, at different times, and/or at different pH. In embodiments, the modified-release formulation comprises a first dose of the AP-based agent and a second dose of the AP-based agent, wherein the first dose and the second dose are released at different locations along the intestines, at different times, and/or at different pH. For example, the first dose is released at the duodenum, and the second dose is released at the ileum. In another example, the first dose is released at the jejunum, and the second dose is released at the ileum. In embodiments, the first dose is released at a location along the small intestine (e.g., the duodenum), while the second dose is released along the large intestine (e.g., the ascending colon). In embodiments, the modified-release formulation may release at least one dose, at least two doses, at least three doses, at least four doses, at least five doses, at least six doses, at least seven doses, or at least eight doses of the AP-based agent at different locations along the intestines, at different times, and/or at different pH.

In embodiments, the formulations of the present disclosure take the form of those as described in one or more of U.S. Pat. Nos. 8,535,713 and 8,911,777 and US Patent Publication Nos. 20120141585, 20120141531, 2006/001896, 2007/0292523, 2008/0020018, 2008/0113031, 2010/0203120, 2010/0255087, 2010/0297221, 2011/0052645, 2013/0243873, 2013/0330411, 2014/0017313, and 2014/0234418, the contents of which are hereby incorporated by reference in their entirety.

In embodiments, the formulations of the present disclosure take the form of those described in one or more of U.S. Pat. Nos. 4,196,564; 4,196,565; 4,247,006; 4,250,997; 4,268,265; 5,317,849; 6,572,892; 7,712,634; 8,074,835; 8,398,912; 8,440,224; 8,557,294; 8,646,591; 8,739,812; 8,810,259; 8,852,631; and 8,911,788 and US Patent Publication Nos. 2014/0302132; 2014/0227357; 20140088202; 20130287842; 2013/0295188; 2013/0307962; and 20130184290, the contents of which are hereby incorporated by reference in their entirety.

In embodiments, the process of formulating the AP-based agent is sufficiently gentle such that the tertiary structure of the AP-based agent (e.g., dimeric structure) is substantially intact. In embodiments, the process of formulating the AP-based agent includes a step of refolding the AP-based agent. In embodiments, the step of refolding the AP-based agent may include the addition of magnesium and/or cyclodextrin.

In embodiments, the modified-release formulation is a modified-release powder formulation.

In embodiments, the modified-release formulation including AP-based agents described herein, and variants thereof, and/or additional therapeutic agents is administered orally.

Suitable dosage forms for oral use include, for example, solid dosage forms such as tablets, capsules, powders, and granules. In embodiments, the modified-release formulation is in the form of powders. In embodiments, the powdered formulations of the present disclosure can be added to food (e.g. juices, strained and/or pureed foods (e.g. fruits, vegetables), sauces, infant formulas, milk, etc.). In embodiments, the modified-release formulation is packaged in the form of a sachet. In embodiments, the modified-release formulation is in the form of tablets. In embodiments, the modified-release formulation is in the form of tablets comprising powders. In embodiments, the modified-release formulation is in the form of capsules. In embodiments, the modified-release formulation is in the form of capsules comprising powders.

In embodiments, the modified-release formulation of the disclosure is in the form of powders. In embodiments, the powders are formed by spray drying and/or by spray-dried dispersion (SDD) technology. In embodiments, the powders comprising AP-based agents are formed by dissolving AP-based agents and polymers in a solvent and then spray-drying the solution. The resulting powder comprises the AP-based agents dispersed within a solid polymeric matrix.

Various types of polymers may be used for the modified-release formulation of the disclosure. In embodiments, the polymer is an enteric polymer that is substantially stable in acidic environments and substantially unstable in near neutral to alkaline environments. In embodiments, the enteric polymer is substantially stable in gastric fluid.

Illustrative polymers include, but are not limited to, copovidone, polyvinyl caprolactam-polyvinyl acetate-polyethyleneglycol copolymer, poly(vinylpyrrolidinone) (PVP), hydroxypropylmethylcellulose or hypromellose (HPMC), hypromellose phthalate (HPMCP), hydroxypropylmethylcellulose or hypromellose acetate succinate (HPMCAS), methacrylate/methacrylic acid copolymer, and mixtures thereof. In embodiments, the polymer is HPMCAS. In embodiments, the poymer is HPMCAS LF, LG, MF, MG, HF, or HG. In embodiments, the polymer is HPMCAS-LF.

### Buffers

Various types of solvents/buffers may be used for preparation of the powders of the disclosure. In embodiments, the solvents/buffers are organic solvents/buffers. Illustrative solvents/buffers that may be used to dissolve the AP-based agent and polymer prior to spray-drying include, but are not limited to, ethanol, methanol, acetone, IPA, tetrahydrafuran, dichloromethane, and mixtures thereof. In embodiments, the solvent used is water such as distilled DI water. In embodiments, the buffer used is sodium phosphate, potassium phosphate, sodium/potassium phosphate, Tris, phosphate, and/or histidine.

In embodiments, enzyme co-factors including zinc and magnesium are used. In embodiments, the enzyme co-factor zinc is used. In embodiments, the zinc is provided as zinc sulfate heptahydrate. In embodiments, the enzyme co-factor magnesium is used. In embodiments, the magnesium is provided as magnesium sulfate heptahydrate.

In embodiments, the formulation includes a protein stabilizer such as trehalose, sucrose, lactose, mannitol, Tween 80, or polyvinyl alcohol. In embodiments, the stabilizer is sucrose. In embodiments, the stabilizer is lactose.

In embodiments, surfactants may be included for the preparation of the powders of the disclosure. The surfactants may be used as solubilizers or emulsifying agents. Illustrative surfactants include, but are not limited to, vitamin E polyethylene glycol succinate, sorbitan monostearate—60/80, polysorbate 20, polysorbate 80, and polyoxyl 40 hydrogenated castor oil.

In embodiments, the powders comprising AP-based agents becomes a gel. In embodiments, the powders comprising an AP-based agent becomes a gel in the intestines. In embodiments, the AP-based agent is released from the gel into one or more regions of the intestines. In embodiments, at pH values greater than about 5 (e.g. about 5, or 6, or 7, or 8, or 9) the gel transforms back into the solution phase and releases the AP enzyme. In embodiments, the gel is used to control the release of the AP-based agent in the intestines. In embodiments, the AP-based agent is released from the gel into one or more of the group consisting of the small intestine, duodenum, jejunum, ileum, large intestine, colon transversum, colon descendens, colon ascendens, colon sigmoidenum, cecum, and rectum.

In embodiments, the formulation of the present disclosure is in the form of powders comprising the AP-based agent dispersed within a solid polymeric matrix. In embodiments, the powders are formed by dissolving AP-based agent and polymers in a solvent to form a solution that is subsequently spray-dried. In embodiments, the solution for spray-drying comprises about 0.1-1% by weight of AP-based agent. For example, the AP-based agent may be present about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, or about 1.0% by weight. In embodiments, the solution comprises about 1-10% by weight a polymer (e.g., HPMCAS-LF). For example, the polymer may be present at about 1%, about 2%, about 3%, about 4%, about 5%, about 6%, about 7%, about 8%, about 9%, or about 10% by weight. In embodiments, the solution comprises about 0.05-0.5% by weight buffer (e.g., monosodium phosphate monohydrate). For example, the buffer may be present at about 0.05%, about 0.06%, about 0.07%, about 0.08%, about 0.09%, about 0.10%, about 0.11%, about 0.12%, about 0.13%, about 0.14%, about 0.15%, about 0.16%, about 0.17%, about 0.18%, about 0.19%, about 0.20%, about 0.25%, about 0.30%, about 0.35%, about 0.40%, about 0.45%, or about 0.50% by weight. In embodiments, the solution comprises about 0.001-0.01% by weight zinc (e.g., zinc sulfate heptahhydrate). For example, the zinc may be present at about 0.001%, about 0.002%, about 0.003%, about 0.004%, about 0.005%, about 0.006%, about 0.007%, about 0.008%, about 0.009%, or about 0.01% by weight. In embodiments, the solution comprises about 0.01-0.1% by weight magnesium (e.g., magnesium sulfate heptahhydrate). For example, the magnesium may be present at about 0.01%, about 0.02%, about 0.03%, about 0.04%, about 0.05%, about 0.06%, about 0.07%, about 0.08%, about 0.09%, or about 0.1% by weight. In embodiments, the solution comprises about 0.1-1% by weight a protein stabilizer (e.g., trehalose). For example, the protein stabilizer may be present at about 0.1%, about 0.2%, about 0.3%, about 0.4%, about 0.5%, about 0.6%, about 0.7%, about 0.8%, about 0.9%, or about 1% by weight. In embodiments, the solution comprises about 90-99.9% by weight solvent (e.g., water). For example, the solvent may be present at about 90%, about 91%, about 92%, about 93%, about 94%, about 95%, about 96%, about 97%, about 98%, or about 99% by weight.

In embodiments, the modified-release formulation of the disclosure is in the form of tablets or capsules. In embodiments, the modified-release formulation is in the form of tablets or capsules comprising the powders of the disclosure. A variety of approaches for generating tablets or capsules may be utilized to include powders of the disclosure. In embodiments, tablets of the disclosure are generated by granulation such as dry granulation. In embodiments, the powders are pre-compressed and the resulting tablet or slug is milled to yield granules.

Alternatively, the powders are pre-compressed with pressure rolls to yield granules. In embodiments, the powders are encapsulated into capsules. In embodiments, the capsule is a gelatin capsule, such as a hard gelatin capsule. In embodiments, the capsule is a hydroxypropyl methylcellulose (HPMC) capsule.

In embodiments, the tablets or capsules comprise a delayed-release coating that includes an enteric agent that is substantially stable in acidic environments and substantially unstable in near neutral to alkaline environments. In embodiments, the delayed-release coating contains an enteric agent that is substantially stable in gastric fluid. The enteric agent can be selected from, for example, solutions or dispersions of methacrylic acid copolymers, cellulose acetate phthalate, hydroxypropylmethyl cellulose phthalate, polyvinyl acetate phthalate, carboxymethylethylcellulose, and EUDRAGIT®-type polymer (poly(methacrylic acid, methylmethacrylate), hydroxypropyl methylcellulose acetate succinate, cellulose acetate trimellitate, shellac or other suitable enteric coating polymers. The polymers are described in international pharmacopeias such as Ph. Eur., USP/NF, DMF, and JPE. The EUDRAGIT®-type polymers include, for example, EUDRAGIT® FS 30D, L 30 D-55, L 100-55, L 100, L 12, 5, L 12, 5 P, RL 30 D, RL PO, RL 100, RL 12, 5, RS 30 D, RS PO, RS 100, RS 12, 5, NE 30 D, NE 40 D, NM 30 D, S 100, S 12, 5, and S 12, 5 P. Similar polymers include Kollicoat® MAE 30 DP and Kollicoat® MAE 100 P. In embodiments, one or more of EUDRAGIT® FS 30D, L 30 D-55, L 100-55, L 100, L 12, 5, L 12, 5 P RL 30 D, RL PO, RL 100, RL 12, 5, RS 30 D, RS PO, RS 100, RS 12, 5, NE 30 D, NE 40 D, NM 30 D, S 100, S 12, 5 S 12, 5 P, Kollicoat® MAE 30 DP and Kollicoat® MAE 100 P is used. In embodiments, the enteric agent may be a combination of the foregoing solutions or dispersions. In embodiments, the delayed-release coating includes the enteric agent EUDRAGIT® L 100. In embodiments, the tablet or capsule is coated with the enteric agent at a coating weight of about 1-20% such as about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% coating weight.

In embodiments, the modified-release formulation of the present disclosure comprises a core particle and a base coat over the core particle, where the base coat comprises the AP-based agent. In embodiments, the core particle comprises sucrose. In embodiments, the AP-based agent of the base coat is encapsulated within the core particle, and can include a plurality of core particles.

In embodiments, the formulation of the present disclosure is in the form of a capsule (e.g., a hard gelatin or HPMC capsule) comprising a plurality of enteric-coated AP-based agent-containing pellets. In embodiments, the pellets (or each individual pellet) comprise an AP-based agent (e.g. IAP, or the other AP-based agent agents described herein, and variants thereof), a sucrose sphere, which the AP-based agent, for example, IAP or a variant, is sprayed onto, a binder excipient (e.g., hydroxypropylcellulose (HPC)), an enteric polymer (e.g., EUDRAGIT L30 D-55), HTP-20 (e.g., PLASACRYL HIP 20), which is an additive that improves coating efficiency and reduces processing times, and buffer salts (e.g., a Tris base, phosphates, magnesium chloride, magnesium sulfate, zinc chloride or zinc sulfate).

In embodiments, the formulation of the present disclosure comprises at least one modified-release pellet, wherein each modified-release pellet comprises about 1-10% by weight AP-based agent (e.g. IAP, or the other AP-based agent agents described herein, and variants thereof). For example, the AP-based agent (e.g. IAP, or the other AP-based agent agents described herein, and variants thereof) may be present at about 1%, about 2%, about 3%, about 4%, about 5%, about 6%, about 7%, about 8%, about 9%, or about 10% by weight. In embodiments, the pellets (or each individual pellet) comprise about 75-95% by weight sucrose sphere. For example, the sucrose sphere may be present at about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95% by weight. In embodiments, the pellets (or each individual pellet) comprise about 5-15% by weight hydroxypropylcellulose (HPC). For example, the hydroxypropylcellulose may be present at about 5%, about 6%, about 7%, about 8%, about 9%, about 10%, about 11%, about 12%, about 13%, about 14%, or about 15% by weight. In embodiments, the pellets (or each individual pellet) comprise about 0.5-2% by weight of buffer salt. The buffer salts may be selected from a Tris base, magnesium chloride, and zinc sulfate. For example, the buffer salts may be present at about 0.5%, about 0.6%, about 0.7%, about 0.8%, about 0.9%, about 1.0%, about 1.1%, about 1.2%, about 1.3%, about 1.4%, about 1.5%, 1.6%, about 1.7%, about 1.8%, about 1.9%, or about 2.0% by weight. In embodiments, the formulation comprises a single layer enteric coating comprising about 20-40%, about 25-40%, about 25-35%, about 30-40%, or about 35-40% enteric polymer weight gain. In embodiments, the formulation comprises a double layer enteric coating comprising about 20-40%, about 25-40%, about 25-35%, about 30-40%, or about 35-40% enteric polymer (e.g., EUDRAGIT L30 D-55) weight gain and about 5-15%, about 5-10%, about 7-15%, about 7-10%, about 10-15%, about 6-9%, or about 7-8% hydroxypropylcellulose weight gain. The weight as described herein refers to the total weight of all components.

In embodiments, the formulation of the present disclosure comprises at least one modified-release pellet, wherein each modified-release pellet comprises about 5% by weight AP-based agent (e.g. IAP, or the other AP-based agent agents described herein, and variants thereof); about 85% by weight sucrose sphere; about 9% by weight hydroxypropylcellulose; and about 1% by weight of buffer salt. In embodiments, the formulation comprises a single layer enteric coating comprising about 20-40% enteric polymer (e.g., EUDRAGIT L30 D-55) weight gain or a double layer enteric coating comprising about 30% enteric polymer (e.g., EUDRAGIT L30 D-55) weight gain and about 7% hydroxypropylcellulose weight gain. The weight as described herein refers to the total weight of all components.

In embodiments, the formulation of the present disclosure comprises at least one modified-release pellet, wherein each modified-release pellet comprises about 4.7% by weight AP-based agent (e.g. IAP, or the other AP-based agent agents described herein, and variants thereof); about 84.9% by weight sucrose sphere; about 9.3% by weight hydroxypropylcellulose; and about 1.2% by weight of buffer salt. In embodiments, the formulation comprises a single layer enteric coating comprising about 20-40% enteric polymer (e.g., EUDRAGIT L 30 D-55) weight gain or a double layer enteric coating comprising about 30% enteric polymer (e.g., EUDRAGIT L 30 D-55) weight gain and about 7% hydroxypropylcellulose weight gain. The weight as described herein refers to the total weight of all components.

In embodiments, the formulation of the present disclosure is in the form of a capsule (e.g., a hard gelatin or HPMC capsule) comprising about 25 mg of the AP-based agent (e.g. IAP, or the other AP-based agent agents described herein, and variants thereof). The capsule includes a plurality of enteric-coated AP-based agent-containing pellets. In embodiments, the formulation of the present disclosure comprises at least one modified-release pellet, wherein each modified-release pellet comprises about 1-15% by weight AP-based agent (e.g. IAP, or the other AP-based agent agents described herein, and variants thereof). For example, the AP-based agent (e.g. IAP, or the other AP-based agent agents described herein, and variants thereof) may be present at about 1%, about 2%, about 3%, about 4%, about 5%, about 6%, about 7%, about 8%, about 9%, about 10%, about 12%, about 13%, about 14%, or about 15% by weight. In embodiments, the pellets (or each individual pellet) comprise about 30-55% by weight sucrose sphere. For example, the sucrose sphere may be present at about 30%, about 31%, about 32%, about 33%, about 34%, about 35%, about 36%, about 37%, about 38%, about 39%, 40%, about 41%, about 42%, about 43%, about 44%, about 45%, about 46%, about 47%, about 48%, about 49%, about 50%, about 51%, about 52%, about 53%, about 54%, or about 55% by weight. In embodiments, the pellets (or each individual pellet) comprise about 5-25% by weight hydroxypropylcellulose (HPC). For example, the hydroxypropylcellulose may be present at about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% by weight. In embodiments, the pellets (or each individual pellet) comprise about 20-35% by weight EUDRAGIT L30 D-55. For example, the EUDRAGIT L30 D-55 may be present at about 20%, about 21%, about 22%, about 23%, about 24%, about 25%, about 26%, about 27%, about 28%, about 29%, about 30%, about 31%, about 32%, about 33%, about 34%, or about 35% by weight. In embodiments, the pellets (or each individual pellet) comprise about 0.5-11% by weight HTP-20. For example, the HTP-20 may be present at about 0.5%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, about 10.5%, or about 11.0% by weight. In embodiments, the pellets (or each individual pellet) comprise about 0.5-2.5% by weight of buffer salt. The buffer salts may be selected from a Tris base, magnesium chloride, magnesium sulfate, zinc chloride and zinc sulfate. For example, the buffer salts may be present at about 0.5%, about 0.6%, about 0.7%, about 0.8%, about 0.9%, about 1.0%, about 1.1%, about 1.2%, about 1.3%, about 1.4%, about 1.5%, 1.6%, about 1.7%, about 1.8%, about 1.9%, about 2.0%, about 2.1%, about 2.2%, about 2.3%, about 2.4%, or about 2.5% by weight.

In embodiments, the formulation of the present disclosure is in the form of a capsule (e.g., a hard gelatin or HPMC capsule) comprising about 25 mg of the AP-based agent (e.g. IAP, or the other AP-based agent agents described herein, and variants thereof). The capsule includes a plurality of enteric-coated AP-based agent-containing pellets. In embodiments, the formulation comprises about 10% by weight AP-based agent (e.g. IAP, or the other AP-based agent agents described herein, and variants thereof); about 38% by weight sucrose sphere; about 19% by weight hydroxypropylcellulose (HPC); about 2% by weight of buffer salt; and about 26% by weight enteric polymer (e.g., EUDRAGIT L30 D-55).

In embodiments, the formulation of the present disclosure is in the form of a capsule (e.g., a hard gelatin or HPMC capsule) comprising about 25 mg of the AP-based agent (e.g. IAP, or the other AP-based agent agents described herein, and variants thereof). The capsule includes a plurality of enteric-coated AP-based agent-containing pellets. In embodiments, the formulation comprises about 9.7% by weight AP-based agent (e.g. IAP, or the other AP-based agent agents described herein, and variants thereof); about 37.7% by weight sucrose sphere; about 19.4% by weight hydroxypropylcellulose (HPC); about 2.4% by weight of buffer salt; and about 26.3% by weight enteric polymer (e.g., EUDRAGIT L 30 D-55).

In embodiments, the formulation of the present disclosure is in the form of a capsule (e.g., a hard gelatin or HPMC capsule) comprising about 5 mg of the AP-based agent (e.g. IAP, or the other AP-based agent agents described herein, and variants thereof). The capsule includes a plurality of enteric-coated AP-based agent-containing pellets. In embodiments, the formulation of the present disclosure comprises at least one modified-release pellet, wherein each modified-release pellet comprises about 1-15% by weight AP-based agent (e.g. IAP, or the other AP-based agent agents described herein, and variants thereof). For example, the AP-based agent (e.g. IAP, or the other AP-based agent agents described herein, and variants thereof) may be present at about 1%, about 2%, about 3%, about 4%, about 5%, about 6%, about 7%, about 8%, about 9%, about 10%, about 12%, about 13%, about 14%, or about 15% by weight. In embodiments, the pellets (or each individual pellet) comprise about 30-55% by weight sucrose sphere. For example, the sucrose sphere may be present at about 30%, about 31%, about 32%, about 33%, about 34%, about 35%, about 36%, about 37%, about 38%, about 39%, 40%, about 41%, about 42%, about 43%, about 44%, about 45%, about 46%, about 47%, about 48%, about 49%, about 50%, about 51%, about 52%, about 53%, about 54%, or about 55% by weight. In embodiments, the pellets (or each individual pellet) comprise about 5-25% by weight hydroxypropylcellulose (HPC). For example, the hydroxypropylcellulose may be present at about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% by weight. In embodiments, the pellets (or each individual pellet) comprise about 20-35% by weight EUDRAGIT L30 D-55. For example, the EUDRAGIT L30 D-55 may be present at about 20%, about 21%, about 22%, about 23%, about 24%, about 25%, about 26%, about 27%, about 28%, about 29%, about 30%, about 31%, about 32%, about 33%, about 34%, or about 35% by weight. In embodiments, the pellets (or each individual pellet) comprise about 0.5-11% by weight HTP-20. For example, the HTP-20 may be present at about 0.5%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, about 10.5%, or about 11.0% by weight. In embodiments, the pellets (or each individual pellet) comprise about 0.5-2.5% by weight of buffer salt. The buffer salts may be selected from a Tris base, magnesium chloride, magnesium sulfate, zinc chloride and zinc sulfate. For example, the buffer salts may be present at about 0.5%, about 0.6%, about 0.7%, about 0.8%, about 0.9%, about 1.0%, about 1.1%, about 1.2%, about 1.3%, about 1.4%, about 1.5%, 1.6%, about 1.7%, about 1.8%, about 1.9%, about 2.0%, about 2.1%, about 2.2%, about 2.3%, about 2.4%, or about 2.5% by weight.

In embodiments, the formulation of the present disclosure is in the form of a capsule (e.g., a hard gelatin or FIPMC capsule) comprising about 5 mg of the AP-based agent (e.g. IAP, or the other AP-based agent agents described herein, and variants thereof). The capsule includes a plurality of enteric-coated AP-based agent-containing pellets. In embodiments, the formulation comprises about 9.7% by weight AP-based agent (e.g. IAP, or the other AP-based agent agents described herein, and variants thereof); about 37.7% by weight sucrose sphere; about 19.4% by weight hydroxypropylcellulose (HPC); about 2.4% by weight of buffer salt; and about 26.3% by weight enteric polymer (e.g., EUDRAGIT L 30 D-55).

In embodiments, the formulation of the present disclosure is in the form of a capsule (e.g., a hard gelatin or FIPMC capsule) comprising about 5 mg of the AP-based agent (e.g. IAP, or the other AP-based agent agents described herein, and variants thereof). The capsule includes a plurality of enteric-coated AP-based agent-containing pellets. In embodiments, the formulation comprises about 9.7% by weight AP-based agent (e.g. IAP, or the other AP-based agent agents described herein, and variants thereof); about 37.7% by weight sucrose sphere; about 19.4% by weight hydroxypropylcellulose (HPC); about 2.4% by weight of buffer salt; and about 26.3% by weight enteric polymer (e.g., EUDRAGIT L 30 D-55).

In embodiments, the formulation of the present disclosure is in the form of a capsule (e.g., a hard gelatin or FIPMC capsule) comprising about 15 mg of the AP-based agent (e.g. IAP, or the other AP-based agent agents described herein, and variants thereof). The capsule includes a plurality of enteric-coated AP-based agent-containing pellets. In embodiments, the formulation of the present disclosure comprises at least one modified-release pellet, wherein each modified-release pellet comprises about 1-15% by weight AP-based agent (e.g. IAP, or the other AP-based agent agents described herein, and variants thereof). For example, the AP-based agent (e.g. IAP, or the other AP-based agent agents described herein, and variants thereof) may be present at about 1%, about 2%, about 3%, about 4%, about 5%, about 6%, about 7%, about 8%, about 9%, about 10%, about 12%, about 13%, about 14%, or about 15% by weight. In embodiments, the pellets (or each individual pellet) comprise about 30-55% by weight sucrose sphere. For example, the sucrose sphere may be present at about 30%, about 31%, about 32%, about 33%, about 34%, about 35%, about 36%, about 37%, about 38%, about 39%, 40%, about 41%, about 42%, about 43%, about 44%, about 45%, about 46%, about 47%, about 48%, about 49%, about 50%, about 51%, about 52%, about 53%, about 54%, or about 55% by weight. In embodiments, the pellets (or each individual pellet) comprise about 5-25% by weight hydroxypropylcellulose (HPC). For example, the hydroxypropylcellulose may be present at about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% by weight. In embodiments, the pellets (or each individual pellet) comprise about 20-35% by weight EUDRAGIT L30 D-55. For example, the EUDRAGIT L30 D-55 may be present at about 20%, about 21%, about 22%, about 23%, about 24%, about 25%, about 26%, about 27%, about 28%, about 29%, about 30%, about 31%, about 32%, about 33%, about 34%, or about 35% by weight. In embodiments, the pellets (or each individual pellet) comprise about 0.5-11% by weight HTP-20. For example, the HTP-20 may be present at about 0.5%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, about 10.5%, or about 11.0% by weight. In embodiments, the pellets (or each individual pellet) comprise about 0.1-2.5% by weight of buffer salt. The buffer salts may be selected from a Tris base, magnesium chloride, magnesium sulfate, zinc chloride and zinc sulfate. For example, the buffer salts may be present at about 0.1%, 0.2%, 0.3%, 0.4%, 0.5%, about 0.6%, about 0.7%, about 0.8%, about 0.9%, about 1.0%, about 1.1%, about 1.2%, about 1.3%, about 1.4%, about 1.5%, 1.6%, about 1.7%, about 1.8%, about 1.9%, about 2.0%, about 2.1%, about 2.2%, about 2.3%, about 2.4%, or about 2.5% by weight.

In embodiments, the formulation of the present disclosure is in the form of a capsule (e.g., a hard gelatin or FI PMC capsule) comprising about 15 mg of the AP-based agent (e.g. IAP, or the other AP-based agent agents described herein, and variants thereof). The capsule includes a plurality of enteric-coated AP-based agent-containing pellets. In embodiments, the formulation comprises about 10% by weight AP-based agent (e.g. IAP, or the other AP-based agent agents described herein, and variants thereof); about 39% by weight sucrose sphere; about 20% by weight hydroxypropylcellulose (HPC); about 0.5% by weight of buffer salt; and about 26% by weight enteric polymer (e.g., EUDRAGIT L30 D-55).

In embodiments, the formulation of the present disclosure is in the form of a capsule (e.g., a hard gelatin or FI PMC capsule) comprising about 15 mg of the AP-based agent (e.g. IAP, or the other AP-based agent agents described herein, and variants thereof). The capsule includes a plurality of enteric-coated AP-based agent-containing pellets. In embodiments, the formulation comprises about 10.0% by weight AP-based agent (e.g. IAP, or the other AP-based agent agents described herein, and variants thereof); about 38.9% by weight sucrose sphere; about 20.0% by weight hydroxypropylcellulose (HPC); about 0.3% by weight of buffer salt; and about 26.3% by weight enteric polymer (e.g., EUDRAGIT L 30 D-55).

In embodiments, the formulation of the present disclosure is in the form of a capsule (e.g., a hard gelatin or HPMC capsule) comprising about 5 mg of the AP-based agent (e.g. IAP, or the other AP-based agent agents described herein, and variants thereof). The capsule includes a plurality of enteric-coated AP-based agent-containing pellets. In embodiments, the formulation of the present disclosure comprises at least one modified-release pellet, wherein each modified-release pellet comprises about 1-15% by weight AP-based agent (e.g. IAP, or the other AP-based agent agents described herein, and variants thereof). For example, the AP-based agent (e.g. IAP, or the other AP-based agent agents described herein, and variants thereof) may be present at about 1%, about 2%, about 3%, about 4%, about 5%, about 6%, about 7%, about 8%, about 9%, about 10%, about 12%, about 13%, about 14%, or about 15% by weight. In embodiments, the pellets (or each individual pellet) comprise about 30-55% by weight sucrose sphere. For example, the sucrose sphere may be present at about 30%, about 31%, about 32%, about 33%, about 34%, about 35%, about 36%, about 37%, about 38%, about 39%, 40%, about 41%, about 42%, about 43%, about 44%, about 45%, about 46%, about 47%, about 48%, about 49%, about 50%, about 51%, about 52%, about 53%, about 54%, or about 55% by weight. In embodiments, the pellets (or each individual pellet) comprise about 5-25% by weight hydroxypropylcellulose (HPC). For example, the hydroxypropylcellulose may be present at about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% by weight. In embodiments, the pellets (or each individual pellet) comprise about 20-35% by weight EUDRAGIT L30 D-55. For example, the EUDRAGIT L30 D-55 may be present at about 20%, about 21%, about 22%, about 23%, about 24%, about 25%, about 26%, about 27%, about 28%, about 29%, about 30%, about 31%, about 32%, about 33%, about 34%, or about 35% by weight. In embodiments, the pellets (or each individual pellet) comprise about 0.5-11% by weight HTP-20. For example, the HTP-20 may be present at about 0.5%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, about 10.5%, or about 11.0% by weight. In embodiments, the pellets (or each individual pellet) comprise about 0.1-2.5% by weight of buffer salt. The buffer salts may be selected from a Tris base, magnesium chloride, magnesium sulfate, zinc chloride and zinc sulfate. For example, the buffer salts may be present at about 0.1%, 0.2%, 0.3%, 0.4%, 0.5%, about 0.6%, about 0.7%, about 0.8%, about 0.9%, about 1.0%, about 1.1%, about 1.2%, about 1.3%, about 1.4%, about 1.5%, 1.6%, about 1.7%, about 1.8%, about 1.9%, about 2.0%, about 2.1%, about 2.2%, about 2.3%, about 2.4%, or about 2.5% by weight.

In embodiments, the formulation of the present disclosure is in the form of a capsule (e.g., a hard gelatin or HPMC capsule) comprising about 5 mg of the AP-based agent (e.g. IAP, or the other AP-based agents described herein, and variants thereof). The capsule includes a plurality of enteric-coated AP-based agent-containing pellets. In embodiments, the formulation comprises about 10% by weight AP-based agent (e.g. IAP, or the other AP-based agent agents described herein, and variants thereof); about 39% by weight sucrose sphere; about 20% by weight hydroxypropylcellulose (HPC); about 0.5% by weight of buffer salt; and about 26% by weight enteric polymer (e.g., EUDRAGIT L30 D-55).

In embodiments, the formulation of the present disclosure is in the form of a capsule (e.g., a hard gelatin or HPMC capsule) comprising about 5 mg of the AP-based agent (e.g. IAP, or the other AP-based agent agents described herein, and variants thereof). The capsule includes a plurality of enteric-coated AP-based agent-containing pellets. In embodiments, the formulation comprises about 10.0% by weight AP-based agent (e.g. IAP, or the other AP-based agent agents described herein, and variants thereof); about 38.9% by weight sucrose sphere; about 20.0% by weight hydroxypropylcellulose (HPC); about 0.3% by weight of buffer salt; and about 26.3% by weight enteric polymer (e.g., EUDRAGIT L 30 D-55).

In embodiments, the administration of the modified-release formulation including AP-based agent (and/or additional therapeutic agents) is any one of oral, intravenous, and parenteral. In embodiments, the administration of the modified-release formulation including AP-based agent (and/or additional agents) is not intravenous in order to, for example, prevent interference with an antibiotic administered systemically. In embodiments, routes of administration include, for example: oral, intradermal, intramuscular, intraperitoneal, intravenous, subcutaneous, intranasal, epidural, sublingual, intracerebral, intravaginal, transdermal, rectally, by inhalation, or topically, particularly to the ears, nose, eyes, or skin.

In embodiments, any formulation of the present disclosure comprises a core particle having a size between about 0.8 mm to about 2.0 mm, between about 0.9 mm to about 1.9 mm, between about 1 mm to about 1.8 mm, between about 1.1 mm to about 1.7 mm, between about 1.2 mm to about 1.6 mm, between about 1.3 mm to about 1.5 mm, between about 1 mm to about 1.3 mm, between about 1 mm to about 1.4 mm, between about 1 mm to about 1.5 mm, between about 1 mm to about 1.6 mm, between about 1 mm to about 1.7 mm, between about 1 mm to about 1.9 mm between about 1 mm to about 2.0 mm diameter. In embodiments, the formulation comprises a core particle having a size of about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, or about 2.0 mm diameter.

Any modified-release formulation including AP-based agent (and/or additional therapeutic agents) as described herein can be administered orally. Such inventive formulations can also be administered by any other convenient route, for example, by intravenous infusion or bolus injection, by absorption through epithelial or mucocutaneous linings (e.g., oral mucosa, rectal and intestinal mucosa, etc.) and can be administered together with an additional therapeutic agent. Administration can be systemic or local. Suitable dosage forms for oral use include, for example, solid dosage forms such as tablets, dispersible powders, granules, and capsules. In embodiments, the modified-release formulation is in the form of a capsule. In embodiments, the modified-release formulation is in the form of a tablet. In embodiments, the modified-release formulation is in the form of a soft-gel capsule. In embodiments, the modified-release formulation is in the form of a gelatin or hydroxypropyl methylcellulose (HPMC) capsule.

Solid oral dosage forms can be prepared by, for example granulation (e.g., wet or dry granulation) of the agents of the disclosure with one or more suitable excipients. Alternatively, the agents of the disclosure can be layered onto an inert core (e.g., a nonpareil/sugar sphere such as a sucrose sphere or silica sphere) using conventional methods such as fluidized bed or pan coating, or extruded and spheronized using methods known in the art, into active compound-containing pellets. In embodiments, the AP-based agent (e.g. IAP, or the other AP-based agent agents described herein, and variants thereof) is spray-coated onto a sucrose sphere. Such pellets can then be incorporated into tablets or capsules using conventional methods.

In embodiments, the present formulation is a modified-release formulation comprising at least one modified-release pellet comprising an IAP. In embodiments, the modified-release formulation releases a substantial amount of the IAP in the GI tract. In embodiments, each modified-release pellet comprises about 1-10% by weight IAP; about 75-95% by weight sucrose sphere; about 5-15% by weight hydroxypropylcellulose; and about 0.5-2% by weight of buffer salt. In embodiments, each modified-release pellet comprises about 5% by weight IAP; about 85% by weight sucrose sphere; about 9% by weight hydroxypropylcellulose; and about 1% by weight of buffer salt. In embodiments, each modified-release pellet comprises about 4.7% by weight IAP; about 84.9% by weight sucrose sphere; about 9.3% by weight hydroxypropylcellulose; and about 1.2% by weight of buffer salt.

### Administration and Dosages

It will be appreciated that the actual dose of the IAP to be administered according to the present disclosure will vary according to the particular compound, the particular dosage form, and the mode of administration. Many factors that may modify the action of the AP-based agent (e.g., body weight, gender, diet, time of administration, route of administration, rate of excretion, condition of the subject, drug combinations, genetic disposition and reaction sensitivities) can be taken into account by those skilled in the art. Administration can be carried out continuously or in one or more discrete doses within the maximum tolerated dose. Optimal administration rates for a given set of conditions can be ascertained by those skilled in the art using conventional dosage administration tests.

Individual doses of the IAP can be administered in unit dosage forms (e.g., tablets or capsules) containing, for example, from about 0.01 mg to about 1,000 mg, about 0.01 mg to about 900 mg, about 0.01 mg to about 800 mg, about 0.01 mg to about 700 mg, about 0.01 mg to about 600 mg, about 0.01 mg to about 500 mg, about 0.01 mg to about 400 mg, about 0.01 mg to about 300 mg, about 0.01 mg to about 200 mg, from about 0.1 mg to about 100 mg, from about 0.1 mg to about 90 mg, from about 0.1 mg to about 80 mg, from about 0.1 mg to about 70 mg, from about 0.1 mg to about 60 mg, from about 0.1 mg to about 50 mg, from about 0.1 mg to about 40 mg, from about 0.1 mg to about 30 mg, from about 0.1 mg to about 20 mg, from about 0.1 mg to about 10 mg, from about 0.1 mg to about 5 mg, from about 0.1 mg to about 3 mg, or from about 0.1 mg to about 1 mg active ingredient per unit dosage for. For example, a unit dosage form can be about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 200 mg, about 300 mg, about 400 mg, about 500 mg, about 600 mg, about 700 mg, about 800 mg, about 900 mg, or about 1,000 mg of the AP-based agent, inclusive of all values and ranges therebetween.

In embodiments, the IAP is administered at an amount of from about 0.01 mg to about 1,000 mg daily, about 0.01 mg to about 900 mg daily, about 0.01 mg to about 800 mg daily, about 0.01 mg to about 700 mg daily, about 0.01 mg to about 600 mg daily, about 0.01 mg to about 500 mg daily, about 0.01 mg to about 400 mg daily, about 0.01 mg to about 300 mg daily, about 0.01 mg to about 200 mg daily, about 0.01 mg to about 100 mg daily, an amount of from about 0.1 mg to about 100 mg daily, from about 0.1 mg to about 95 mg daily, from about 0.1 mg to about 90 mg daily, from about 0.1 mg to about 85 mg daily, from about 0.1 mg to about 80 mg daily, from about 0.1 mg to about 75 mg daily, from about 0.1 mg to about 70 mg daily, from about 0.1 mg to about 65 mg daily, from about 0.1 mg to about 60 mg daily, from about 0.1 mg to about 55 mg daily, from about 0.1 mg to about 50 mg daily, from about 0.1 mg to about 45 mg daily, from about 0.1 mg to about 40 mg daily, from about 0.1 mg to about 35 mg daily, from about 0.1 mg to about 30 mg daily, from about 0.1 mg to about 25 mg daily, from about 0.1 mg to about 20 mg daily, from about 0.1 mg to about 15 mg daily, from about 0.1 mg to about 10 mg daily, from about 0.1 mg to about 5 mg daily, from about 0.1 mg to about 3 mg daily, from about 0.1 mg to about 1 mg daily, or from about 5 mg to about 80 mg daily. In embodiments, the IAP is administered at a daily dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 200 mg, about 300 mg, about 400 mg, about 500 mg, about 600 mg, about 700 mg, about 800 mg, about 900 mg, or about 1,000 mg, inclusive of all values and ranges therebetween.

In embodiments, a suitable dosage of the IAP is in a range of about 0.01 mg/kg to about 100 mg/kg of body weight of the subject, about 0.01 mg/kg to about 90 mg/kg of body weight of the subject, about 0.01 mg/kg to about 80 mg/kg of body weight of the subject, about 0.01 mg/kg to about 70 mg/kg of body weight of the subject, about 0.01 mg/kg to about 60 mg/kg of body weight of the subject, about 0.01 mg/kg to about 50 mg/kg of body weight of the subject, about 0.01 mg/kg to about 40 mg/kg of body weight of the subject, about 0.01 mg/kg to about 30 mg/kg of body weight of the subject, about 0.01 mg/kg to about 20 mg/kg of body weight of the subject, about 0.01 mg/kg to about 10 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, about 20 mg/kg body weight, about 30 mg/kg body weight, about 40 mg/kg body weight, about 50 mg/kg body weight, about 60 mg/kg body weight, about 70 mg/kg body weight, about 80 mg/kg body weight, about 90 mg/kg body weight, or about 100 mg/kg body weight, inclusive of all values and ranges therebetween. In embodiments, a suitable dosage of the AP-based agent is in a range of about 0.01 mg/kg to about 10 mg/kg of body weight, in a range of about 0.01 mg/kg to about 9 mg/kg of body weight, in a range of about 0.01 mg/kg to about 8 mg/kg of body weight, in a range of about 0.01 mg/kg to about 7 mg/kg of body weight, in a range of 0.01 mg/kg to about 6 mg/kg of body weight, in a range of about 0.05 mg/kg to about 5 mg/kg of body weight, in a range of about 0.05 mg/kg to about 4 mg/kg of body weight, in a range of about 0.05 mg/kg to about 3 mg/kg of body weight, in a range of about 0.05 mg/kg to about 2 mg/kg of body weight, in a range of about 0.05 mg/kg to about 1.5 mg/kg of body weight, or in a range of about 0.05 mg/kg to about 1 mg/kg of body weight.

In accordance with certain embodiments of the disclosure, the IAP may be administered, for example, more than once daily (e.g., about two, about three, about four, about five, about six, about seven, about eight, about nine, or about ten times per day), about once per day, about every other day, about every third day, about once a week, about once every two weeks, about once every month, about once every two months, about once every three months, about once every six months, or about once every year.

### Additional Therapeutic Agents and Combination Therapy

Administration of the present compositions and formulations comprising the IAP may be combined with additional therapeutic agents. Co-administration of the additional therapeutic agent and the present compositions/formulations may be simultaneous or sequential. Further, the present compositions/formulations may comprise an additional therapeutic agent (e.g. via co-formulation). For example, the additional therapeutic agent and the IAP may be combined into a single formulation. Alternatively, the additional therapeutic agent and the IAP may be formulated separately.

In embodiments, the additional therapeutic agent and the IAP are administered to a subject simultaneously. The term “simultaneously” as used herein, means that the additional therapeutic agent and the IAP are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and the IAP can be by simultaneous administration of a single formulation (e.g., a formulation comprising the additional therapeutic agent and the IAP) or of separate formulations (e.g., a first formulation including the additional therapeutic agent and a second formulation including the IAP).

In embodiments, the additional therapeutic agent and the IAP are administered to a subject simultaneously but the release of the additional therapeutic agent and the IAP from their respective dosage forms (or single unit dosage form if co-formulated) may occur sequentially.

Co-administration does not require the additional therapeutic agent and the IAP to be administered simultaneously, if the timing of their administration is such that the pharmacological activities of the additional therapeutic agent and the IAP overlap in time. For example, the additional therapeutic agent and the IAP can be administered sequentially. The term “sequentially” as used herein means that the additional therapeutic agent and the IAP are administered with a time separation of more than about 60 minutes. For example, the time between the sequential administration of the additional therapeutic agent and the IAP can be more than about 60 minutes, more than about 2 hours, more than about 5 hours, more than about 10 hours, more than about 1 day, more than about 2 days, more than about 3 days, or more than about 1 week apart. The optimal administration times will depend on the rates of metabolism, excretion, and/or the pharmacodynamic activity of the additional therapeutic agent and the IAP being administered. Either the additional therapeutic agent or the IAP may be administered first.

Co-administration also does not require the additional therapeutic agent and the IAP to be administered to the subject by the same route of administration. Rather, each therapeutic agent can be administered by any appropriate route, for example, parenterally or non-parenterally.

### Definitions

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50%” covers the range of 45% to 55%.

An “effective amount,” when used in connection with medical uses is an amount that is effective for providing a measurable treatment, prevention, or reduction in the rate of pathogenesis of a disorder of interest.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

The amount of compositions described herein needed for achieving a therapeutic effect may be determined empirically in accordance with conventional procedures for the particular purpose. Generally, for administering therapeutic agents (e.g., microbiome-modulating agents and/or additional therapeutic agents described herein) for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures, tissue samples, tissue homogenates or experimental animals, e.g., for determining the LD50 (the dose lethal to about 50% of the population) and the ED50 (the dose therapeutically effective in about 50% of the population). The dosage can vary depending upon the dosage form employed and the route of administration utilized. The dose ratio between toxic and therapeutic effects is the therapeutic index and can be expressed as the ratio LD50/ED50. In embodiments, compositions and methods that exhibit large therapeutic indices are preferred. A therapeutically effective dose can be estimated initially from in vitro assays, including, for example, cell culture assays or measurements or methane production in stool samples. Also, a dose can be formulated in animal models to achieve a circulating plasma concentration range that includes the IC50 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In embodiments, the effect will result in a quantifiable change of at least about 10%, at least about 20%, at least about 30%, at least about 50%, at least about 70%, or at least about 90%. In embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein.

### EXAMPLES

### Example 1: Measurement of Trans Epithelial Electric Resistance (TEER) after Administration of IAP

CaCo2 cells form monolayers that exhibit tight junctions between adjacent cells. IAP reduces the flux of compounds (e.g. ions, Lucifer Yellow) through a cell monolayer that comprises tight junctions. IAP also exhibits the ability to increase TEER across a cell monolayer that comprises tight junctions. Treatment of CaCo2 monolayers with IAP leads to decreased Lucifer Yellow permeability through CaCo2 monolayers compared to vehicle alone. Treatment of CaCo2 monolayers with IAP leads to an increase in TEER across CaCo2 monolayers compared to vehicle alone.

### Example 2: In Vivo Efficacy of AP-Based Agents in Treatment in Models of Celiac Disease

An AP-based agent, including bIAP (without limitation, a composition having the enzyme of SEQ ID NO: 11, or SEQ ID Nos: 1-10, or a variant thereof).

The Model to be used is chosen from a murine model (e.g. a mouse model that reproduces the dual overexpression of IL-15 in the gut epithelium and the lamina propria (LP) characteristic of active celiac disease, expresses the predisposing HLA-DQ8 molecule, and develops villous atrophy (VA) upon gluten ingestion as in Nature. 2020 February; 578(7796): 600-604, the entire contents of which are hereby incorporated by reference); human gut derived-organoids (e.g. intestinal organoids developed from duodenal biopsies from both non-celiac (NC) and celiac (CD) patients, as in Scientific Reports volume 9, Article number: 7029 (2019), the entire contents of which are hereby incorporated by reference); rhesus macaque (as in PLOS One. 2008; 3(2):e1614, the entire contents of which are hereby incorporated by reference); or a model as described in Int Rev Immunol. 2011 August; 30(4): 197-206, the entire contents of which are hereby incorporated by reference.

By way of non-limitation, one of the above models is used in a treatment or prevention (prophylaxis) study). Animals are provided standard diets or gluten free diets, as appropriate. Animals are administered the AP-agent to diseased animals or animals to be challenged with disease.

For instance, in the murine model above, H&E staining of paraffin-embedded ileum sections and/or transcriptional analysis is undertaken (e.g. comparison of the intestinal epithelium and lamina propria (LP) in gluten-fed and/or normal fed and/or AP-treated and/or sham-treated animals).

Any of the methods of Nature 2020 February; 578(7796): 600-604, the entire contents of which are hereby incorporated by reference, may be used to assay the celiac treatment or prevention effects of an AP-agent described herein.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A method of treating or preventing celiac disease in a patient in need thereof, comprising administering to the patient a composition comprising an alkaline phosphatase (AP)-based agent, optionally intestinal alkaline phosphatase (IAP).

2. The method of claim 1, wherein the patient is on a gluten-free diet, including only unintentional exposure to gluten.

3. The method of claim 1 or 2, wherein the patient has celiac disease that is nonresponsive or refractory to a gluten-free diet.

4. The method of any one of the above claims, wherein the patient has one or more symptoms selected from abdominal cramping, abdominal pain, bloating, gas, diarrhea, loose stools, nausea, vomiting, indigestion, reflux, constipation, vomiting, headache, and tiredness.

5. The method of any one of the above claims, wherein the patient has low expression and/or activity of IAP in the patient's mucosa.

6. The method of any one of the above claims, wherein the patient is characterized as having low expression and/or activity of IAP by assaying a biological sample from the subject.

7. The method of claim 6, wherein the biological sample is selected from stool, mucus, tissue, blood, plasma, serum, pus, urine, perspiration, tears, sputum, saliva, and/or other body fluids.

8. The method of any one of the above claims, wherein the patient is experiencing one or more Celiac Disease Patient Reported Outcome (CeD PRO) abdominal domain symptoms selected from abdominal pain, abdominal cramping, bloating or gas, and/or one or more CeD PRO GI domain symptoms selected from abdominal cramping, abdominal pain, bloating, gas, diarrhea, loose stools, nausea, indigestion, reflux, constipation, and vomiting, and the patient is experiencing at least one CeD PRO non-GI domain symptom.

9. The method of claim 8, wherein the CeD PRO non-GI domain symptom(s) are one or more of headache or tiredness.

10. The method of any one of the above claims, wherein the patient is experiencing one or more CeD PRO Symptomatic days of at least 2, 3, 4, 5, 6, or 7, wherein a CeD PRO Symptomatic days is a day where the mean of abdominal cramping, abdominal pain, bloating, and gas is scored as ≥2.5 or ≥3 out of a 0 to 10 scale, or a day where the mean of diarrhea and loose stool is scored as ≥2.5 or ≥3 out of a 0 to 10 scale, or a day where nausea is scored as ≥2.5 or ≥3 out of a 0 to 10 scale.

11. The method of claim 10, wherein upon treatment the patient experiences one or more CeD PRO Improved Symptom days per week, wherein a CeD PRO Improved Symptom day is a day where the mean of abdominal cramping, abdominal pain, bloating, and gas is scored as ≤1.5 out of a 0 to 10 scale, and a day where the mean of diarrhea and loose stool is scored as ≤1.5 out of a 0 to 10 scale, and a day where nausea is scored as ≤1 out of a 0 to 10 scale.

12. The method of any one of the above claims, wherein the patient scores at least 2, or at least 3, or at least 4, or at least 5, or at least 6 on the CeD Gastrointestinal Symptom Rating Scale (CeD GSRS) at the start of treatment using the 1-7 Likert scale.

13. The method of any one of the above claims, wherein the patient scores at least 2, or at least 3, or at least 4, or at least 5, or at least 6, or 7 on the Total Gastrointestinal Symptom Rating Scale (GSRS) at the start of treatment using the 1-7 Likert scale.

14. The method of any one of the above claims, wherein the patient experiences at least about 3, 4, 5, 6, 7, 8, 9, 10, or more bowel movements per day at the start of treatment.

15. The method of any one of the above claims, wherein the patient experiences at least about 4, at least about 5, at least about 6, at least about 7, or at least about 8, or at least about 9, or at least about 10 diarrhea or loose stools per day on the Bristol Form Scale (BSFS) at the start of treatment.

16. The method of any one of the above claims, wherein the patient experiences 3 diarrhea or loose stools per day with a score of 5-7 as measured by the Bristol Form Scale (BSFS) at the start of treatment.

17. The method of any one of the above claims, wherein the composition comprising IAP is administered for at least about 9 weeks, or at least about 10 weeks, or at least about 12 weeks.

18. The method of any one of the above claims, wherein the composition comprising IAP is administered prior to meals.

19. The method of any one of the above claims, wherein the IAP is selected from human IAP or calf/bovine IAP.

20. The method of any one of the above claims, wherein the IAP comprises an amino acid sequence having at least about 60%, or at least about 65%, or at least about 70%, or at least about 75%, or at least about 80%, or at least about 85%, or at least about 90%, or at least about 95%, or at least about 96%, or at least about 97%, or at least about 98%, or at least about 99%, or at least about 100% identity with any one of SEQ ID NOs: 11, 1-6 or 10.

21. The method of any one of the above claims, wherein the IAP comprises an amino acid sequence having at least about 60%, or at least about 65%, or at least about 70%, or at least about 75%, or at least about 80%, or at least about 85%, or at least about 90%, or at least about 95%, or at least about 96%, or at least about 97%, or at least about 98%, or at least about 99%, or at least about 100% identity with SEQ ID NO: 2.

22. The method of any one of the above claims, wherein the composition comprising IAP is administered orally.

23. The method of any one of the above claims, wherein the composition comprising IAP is formulated for GI release.

24. The method of any one of the above claims, wherein the bIAP comprises an amino acid sequence having at least about 60%, or at least about 65%, or at least about 70%, or at least about 75%, or at least about 80%, or at least about 85%, or at least about 90%, or at least about 95%, or at least about 96%, or at least about 97%, or at least about 98%, or at least about 99% sequence identity to SEQ ID NO: 11.

25. The method of claim 24, wherein the bIAP comprises an amino acid sequence having at least about 97% sequence identity to SEQ ID NO: 11.

26. The method of claim 25, wherein the bIAP comprises the amino acid sequence of SEQ ID NO: 11.

27. A method of treating celiac disease in a patient in need thereof, comprising administering to the patient a composition comprising an bIAP comprising an amino acid sequence of SEQ ID NO: 11, wherein the IAP is formulated for GI release.


SEQUENCE LISTING (14 sequences)

SEQ ID NO: 1 - protein, 528 aa, Artificial Sequence
  Synthetic polymer.
MQGPWVLLLLGLRLQLSLGVIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEIHRDPTLDPSLMEMTEAALRLLSRNPRGFYLFVEGGRIDHGHHEGVAYQALTEAVMFDDAIERAGQLTSEEDTLTLVTADHSHVFSFGGYTLRGSSIFGLAPSKAQDSKAYTSILYGNGPGYVFNSGVRPDVNESESGSPDYQQQAAVPLSSETHGGEDVAVFARGPQAHLVHGVQEQSFVAHVMAFAACLEPYTACDLAPPACTTDAAHPVAASLPLLAGTLLLLGASAAP

SEQ ID NO: 2 - protein, 533 aa, Artificial Sequence
  Synthetic polymer.
MQGACVLLLLGLHLQLSLGLIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPDAAHLAASPPPLALLAGAMLLLLAPTLY

SEQ ID NO: 3 - protein, 503 aa, Artificial Sequence
  Synthetic polymer.
MQGPWVLLLLGLRLQLSLGVIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEIHRDPTLDPSLMEMTEAALRLLSRNPRGFYLFVEGGRIDHGHHEGVAYQALTEAVMFDDAIERAGQLTSEEDTLTLVTADHSHVFSFGGYTLRGSSIFGLAPSKAQDSKAYTSILYGNGPGYVFNSGVRPDVNESESGSPDYQQQAAVPLSSETHGGEDVAVFARGPQAHLVHGVQEQSFVAHVMAFAACLEPYTACDLAPPAGTTD

SEQ ID NO: 4 - protein, 506 aa, Artificial Sequence
  Synthetic polymer.
MQGACVLLLLGLHLQLSLGLIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPD

SEQ ID NO: 5 - protein, 754 aa, Artificial Sequence
  Synthetic polymer.
MQGACVLLLLGLHLQLSLGLIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPDGGSGGSGGGGSGGGGSEPKSCDKTHTCPPCPAPELLGGPSVFLFPPKPKDTLMISRTPEVTCVVVDVSHEDPQVKFNWYVDGVQVHNAKTKPREQQYNSTYRVVSVLTVLHQNWLDGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 534 aa, Artificial Sequence
  Synthetic polymer.
MQWACVLLLLGLWLQLSLTFIPAEEEDPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYKTIGVSAAARYNQCNTTSGNEVTSVMNRAKKAGKSVGVVTTSRVQHASPAGAYAHTVNRNWYSDADLPADAQTYGCQDIATQLVNNMDIDVILGGGRMYMFPEGTPDPEYPYDVNQTGVRKDKRNLVQEWQAKHQGAQYVWNRTELLQAANDPSVTHLMGLFEPADMKYNVQQDPTKDPTLEEMTEAALQVLSRNPQGFYLFVEGGRIDHGHHEGKAYMALTDTVMFDNAIAKANELTSELDTLILATADHSHVFSFGGYTLRGTSIFGLAPSKASDNKSYTSILYGNGPGYVLGGGLRPDVNDSISEDPSYRQQAAVPLSSESHGGEDVAVFARGPQAHLVHGVQEETFVAHVMAFAGCVEPYTDCNLAPPAGTTDAAHPIEGRSVVPALLPLRAGTLLLLETATAP

SEQ ID NO: 7 - DNA, 1669 bp, Artificial Sequence
  Synthetic polymer.
atgcaggggccctgggtgctgctgctgctgggcctgaggctacagctctccctgggcgtcatcccaggtaatgaggctccccaagctgttccacacacagggcaccccctcagccaggctgacctgatctctactctccccctggccagctgaggaggagaacccggccttctggaaccgccaggcagctgaggccctggatgctgccaagaagctgcagcccatccagaaggtcgccaagaacctcatcctcttcctgggcgatgggttgggggtgcccacggtgacagccaccaggatcctaaaggggcagaagaatggcaaactggggcctgagacgcccctggccatggaccgcttcccatacctggctctgtccaagacatacaatgtggacagacaggtgccagacagcgcagccacagccacggcctacctgtgcggggtcaaggccaacttccagaccatcggcttgagtgcagccgcccgctttaaccagtgcaacacgacacgcggcaatgaggtcatctccgtgatgaaccgggccaagcaagcaggaaagtcagtaggagtggtgaccaccacacgggtgcagcacgcctcgccagccggcacctacgcacacacagtgaaccgcaactggtactcagatgctgacatgcctgcctcagcccgccaggaggggtgccaggacatcgccactcagctcatctccaacatggacattgacgtgatccttggcggaggccgcaagtacatgtttcccatggggaccccagaccctgagtacccagctgatgccagccagaatggaatcaggctggacgggaagaacctggtgcaggaatggctggcaaagcaccagggtgcctggtatgtgtggaaccgcactgagctcatgcaggcgtccctggaccagtctgtgacccatctcatgggcctctttgagcccggagacacgaaatatgagatccaccgagaccccacactggacccctccctgatggagatgacagaggctgccctgcgcctgctgagcaggaacccccgcggcttctacctctttgtggagggcggccgcatcgaccatggtcatcatgagggtgtggcttaccaggcactcactgaggcggtcatgttcgacgacgccattgagagggcgggccagctcaccagcgaggaggacacgctgaccctcgtcaccgctgaccactcccatgtcttctcctttggtggctacaccttgcgagggagctccatcttcgggttggcccccagcaaggctcaggacagcaaagcctacacgtccatcctgtacggcaatggcccgggctacgtgttcaactcaggcgtgcgaccagacgtgaatgagagcgagagcgggagccccgattaccagcagcaggcggcggtgcccctgtcgtccgagacccacggaggcgaagacgtggcggtgtttgcgcgcggcccgcaggcgcacctggtgcatggtgtgcaggagcagagcttcgtagcgcatgtcatggccttcgctgcctgtctggagccctacacggcctgcgacctggcgcctcccgcctgcaccaccgacgccgcgcacccagttgccgcgtcgctgccactgctggccgggaccctgctgctgctgggggcgtccgctgctccctga

SEQ ID NO: 8 - DNA, 1671 bp, Artificial Sequence
  Synthetic polymer.
atgcaggggccctgggtgctgctgctgctgggcctgaggctacagctctccctgggcgtcatcccagctgaggaggagaacccggccttctggaaccgccaggcagctgaggccctggatgctgccaagaagctgcagcccatccagaaggtcgccaagaacctcatcctcttcctgggcgatgggttgggggtgcccacggtgacagccaccaggatcctaaaggggcagaagaatggcaaactggggcctgagacgcccctggccatggaccgcttcccatacctggctctgtccaagacatacaatgtggacagacaggtgccagacagcgcagccacagccacggcctacctgtgcggggtcaaggccaacttccagaccatcggcttgagtgcagccgcccgctttaaccagtgcaacacgacacgcggcaatgaggtcatctccgtgatgaaccgggccaagcaagcaggaaagtcagtaggagtggtgaccaccacacgggtgcagcacgcctcgccagccggcacctacgcacacacagtgaaccgcaactggtactcagatgctgacatgcctgcctcagcccgccaggaggggtgccaggacatcgccactcagctcatctccaacatggacattgacgtgatccttggcggaggccgcaagtacatgtttcccatggggaccccagaccctgagtacccagctgatgccagccagaatggaatcaggctggacgggaagaacctggtgcaggaatggctggcaaagcaccagggtgcctggtatgtgtggaaccgcactgagctcatgcaggcgtccctggaccagtctgtgacccatctcatgggcctctttgagcccggagacacgaaatatgagatccaccgagaccccacactggacccctccctgatggagatgacagaggctgccctgcgcctgctgagcaggaacccccgcggcttctacctctttgtggagggcggccgcatcgaccatggtcatcatgagggtgtggcttaccaggcactcactgaggcggtcatgttcgacgacgccattgagagggcgggccagctcaccagcgaggaggacacgctgaccctcgtcaccgctgaccactcccatgtcttctcctttggtggctacaccttgcgagggagctccatcttcgggttggcccccagcaaggctcaggacagcaaagcctacacgtccatcctgtacggcaatggcccgggctacgtgttcaactcaggcgtgcgaccagacgtgaatgagagcgagagcgggagccccgattaccagcagcaggcggcggtgcccctgtcgtccgagacccacggaggcgaagacgtggcggtgtttgcgcgcggcccgcaggcgcacctggtgcatggtgtgcaggagcagagcttcgtagcgcatgtcatggccttcgctgcctgtctggagccctacacggcctgcgacctggcgcctcccgcctgcaccaccgacgccgcgcacccagttgccgcgtcgctgccactgctggccgggaccctgctgctgctgggggcgtccgctgctccctgatttactaaaaccttgaaataaaattgtaaaacatcagtttgaaggcctgactctcagggtagttcttttttaattctgggtttt

SEQ ID NO: 9 - DNA, 1259 bp, Artificial Sequence
  Synthetic polymer.
aaaaaacaagacaaagctgagatcagaaatgtcattgtgatgataggcgacggcatggggacgccttacataagagcctaccgttccatgaaaaataacggtgacacaccgaataacccgaagttaacagaatttgaccggaacctgacaggcatgatgatgacgcatccggatgaccctgactataatattacagattcagcagcagccggaacagcattagcgacaggcgttaagacatataacaatgcaattggcgtcgataaaaacggaaaaaaagtgaaatctgtacttgaagaggccaaacagcaaggcaagtcaacagggcttgtcgccacgtctgaaattaaccacgccactccagccgcatatggcgcccacaatgaatcacggaaaaacatggaccaaatcgccaacagctatatggatgacaagataaaaggcaaacataaaatagacgtgctgctcggcggcggaaaatcttattttaaccgcaagaacagaaacttgacaaaggaattcaaacaagccggctacagctatgtgacaactaaacaagcattgaaaaaaaataaagatcagcaggtgctcgggcttttcgcagatggagggcttgctaaagcgctcgaccgtgacagtaaaacaccgtctctcaaagacatgacggtttcagcaattgatcgcctgaaccaaaataaaaaaggatttttcttgatggtcgaagggagccagattgactgggcggcccatgacaatgatacagtaggagccatgagcgaggttaaagattttgaacaggcctataaagccgcgattgaatttgcgaaaaaagacaaacatacacttgtgattgcaactgctgaccatacaaccggcggctttaccattggcgcaaacggggaaaagaattggcacgcagaaccgattctctccgctaagaaaacacctgaattcatggccaaaaaaatcaggaaggcaagccggttaaagatgtgctcgcccgctatgccaatctgaaagtcacatctgaagaaatcaaaagcgttgaagcagctgcacaggctgacaaaagcaaaggggcctccaaagccatcatcaagatttttaatacccgctccaacagcggatggacgagtaccgatcataccggcgaagaagtaccggtatacgcgtacggccccggaaaagaaaaattccgcggattgattaacaatacggaccaggcaaacatcatatttaagattttaaaaactggaaaa

SEQ ID NO: 10 - protein, 420 aa, Artificial Sequence
  Synthetic polymer.
KKQDKAEIRNVIVMIGDGMGTPYIRAYRSMKNNGDTPNNPKLTEFDRNLTGMMMTHPDDPDYNITDSAAAGTALATGVKTYNNAIGVDKNGKKVKSVLEEAKQQGKSTGLVATSEINHATPAAYGAHNESRKNMDQIANSYMDDKIKGKHKIDVLLGGGKSYFNRKNRNLTKEFKQAGYSYVTTKQALKKNKDQQVLGLFADGGLAKALDRDSKTPSLKDMTVSAIDRLNQNKKGFFLMVEGSQIDWAAHDNDTVGAMSEVKDFEQAYKAAIEFAKKDKHTLVIATADHTTGGFTIGANGEKNWHAEPILSAKKTPEFMAKKISEGKPVKDVLARYANLKVTSEEIKSVEAAAQADKSKGASKAIIKIFNTRSNSGWTSTDHTGEEVPVYAYGPGKEKFRGLINNTDQANIIFKILKTGK

SEQ ID NO: 11 - protein, 487 aa, Artificial Sequence
  Synthetic polymer.
LIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPD

SEQ ID NO: 12 - protein, 10 aa, Artificial Sequence
  Synthetic polymer.
ALLPLLAGTL

SEQ ID NO: 13 - protein, 10 aa, Artificial Sequence
  Synthetic polymer.
ALLPLRAGTL

SEQ ID NO: 14 - DNA, 13 bp, Artificial Sequence
  Synthetic polymer.
gccgccaccatgg